(19) United States
(12) Patent Application Publication
(10) Pub. No.: US 2016/0252506 A1
(43) Pub. Date: Sep. 1, 2016

(54) SILICON NANOWIRE-BASED SENSOR ARRAYS

(71) Applicant: MICHIGAN TECHNOLOGICAL UNIVERSITY, Houghton, MI (US)

(72) Inventors: Thomas Daunais, Ann Arbor, MI (US); Paul L. Bergstrom, Houghton, MI (US)

(21) Appl. No.: 15/035,595

(22) PCT Filed: Nov. 13, 2014
(86) PCT No.: PCT/US14/65403

§ 371 Date: May 10, 2016

Related U.S. Application Data
(60) Provisional application No. 61/903,686, filed on Nov. 13, 2013.

(51) Int. Cl. G01N 33/543; H01L 21/311; H01L 21/306; H01L 29/06; G01N 33/552; H01L 21/027; H01L 21/308; H01L 29/66; H01L 29/775; G01N 33/569; H01L 21/02; H01L 29/16

(52) U.S. Cl. CPC G01N 33/54386; H01L 21/0217; H01L 21/311; H01L 21/30604; H01L 21/02164; H01L 29/0673; H01L 29/16; H01L 21/02266; H01L 21/02236; H01L 21/0273; H01L 21/3081; H01L 21/3086; H01L 29/66439; H01L 29/775; G01N 33/56916; G01N 33/552; H01L 21/02255

(57) ABSTRACT

A method for fabricating silicon nanowires. The method includes the steps of: depositing a silicon nitride layer on a silicon on insulator (SOI) starting wafer; patterning the silicon nitride to define at least one silicon microbar; etching the SOI starting wafer to expose the at least one silicon microbar, wherein the at least one microbar is surrounded by a raised perimeter; growing a silicon oxide layer on the raised perimeter of the at least one microbar; and etching a portion of the at least one silicon microbar to produce at least one silicon nanowire adjacent the silicon oxide layer.

### CROSS-REFERENCE TO RELATED APPLICATIONS

This application claims priority to co-pending U.S. Provisional Patent Application No. 61/903,686 filed on Nov. 13, 2013, the entire content of which is incorporated herein by reference.

### STATEMENT REGARDING FEDERALLY-SPONSORED RESEARCH OR DEVELOPMENT

This invention was made with government support under DE-SC0005162 awarded by the Department of Energy. The government has certain rights in the invention.

### BACKGROUND

The present invention relates to silicon nanowire sensors for detection of target analytes and methods for making the same.

In the United States, expected lifespan has been prolonged thanks to advances in modern medicine. With prolonged life, the rise in degenerative diseases has been observed; many of these diseases were present in insignificant numbers just decades ago. Currently, the most noted trend is the increase in many forms of cancer, many of which remain undetectable in early stages. Perhaps one of the more challenging statistics alongside the growing cancer rate is that 30% of cancer patients die directly from cachexia, a degenerative disease that causes significant weight loss, muscle atrophy and weakness. Cachexia is not only limited to cancer, it also coincides with AIDS, emphysema, heart failure and kidney failure. There currently is no method to directly detect cachexia; this issue needs to be resolved because there are drugs available to help treat this disease. The key to direct disease detection for cachexia and many other diseases could lie in micro ribonucleic acid microRNA detection.

microRNAs, which were discovered in 1993, are short nucleotides that are tissue-specific, allowing for detection methods to identify damaged tissue by discovery of displaced microRNA in other parts of the body. Currently, there have been several hundred microRNAs identified that can be used as disease markers; but testing for them requires very time-consuming and costly processing. For example, miR-1 has been identified as a marker for cachexia. A new method needs to be developed to sense for microRNA to open the doors to early detection of many diseases that cannot be detected using current techniques.

The medical diagnostic field has limited ability to sense many analytes of interest. Of those that can be sensed currently, including miR-1, many measurements require processing that may take multiple days and even up to one week to obtain conclusive results. In fact, determining susceptibilities of several bacteria such as mycobacteria to antibiotics in many cases may require several days to ascertain. One of the most sensitive methods to identify analytes of interest currently available to the medical industry is a label detection method called fluorescent tagging. In this method, a fluorescent molecule is bound to a chemical that binds with the analyte of interest. After this bond is made, the sample is then put in an instrument such as a photomultiplier to detect the presence of the fluorescent tag. This procedure requires multiple processing steps to prepare the sample that is to be measured, allowing for the possibility of sample contamination. Even with this technique, it is not possible to detect many analytes of interest because the method lacks the ultimate resolution required for very low concentration detection. The key to the next generation of medical sensing technology is to increase the ultimate resolution of the testing method.

In 2012 in the United States, approximately 50 million people experienced a food-borne illness. A significant number of these illnesses were traced back to E. coli contamination of food. This resulted in an estimated 130,000 hospitalizations and 3,000 deaths. Organic farms, farmers markets, and food imports have added substantially to America's table to meet increased demand, but this expansion of food sources will require a matching ramp-up in inspections. In 2009, the FDA estimated 24 million shipments of FDA-regulated goods passed through the nation's ports of entry, up from 6 million a decade earlier. During that time, the number of FDA investigators stayed constant at about 1,350. Antiquated food testing methods and overworked inspectors ensure that only a small percentage of the U.S. food supply is tested for pathogen contamination.

To help meet the regulation requirements for testing, many large food production houses perform in-house testing on their own products. Currently utilized testing methods require one week for conclusive results because they require slow laboratory based testing methods including culture growth prior to testing. Many of these tests occur at labs off-site from the 28,000 food processing facilities in the U.S., adding delay and cost. The industry would benefit from sensing devices that can be deployed on site.

### SUMMARY

Thus, there currently exists an unfulfilled need for an easy to use, rapid diagnostic sensing apparatus with both high selectivity and sensitivity such as a so-called ‘lab-on-a-chip’ sensor. Lab-on-a-chip sensors utilizing nanostructure technologies offer several advantages, including elimination of the majority of processing steps, higher ultimate resolution, and in general a lower cost to operate when compared to their laboratory-based counterparts.

In one aspect, a method for fabricating silicon nanowires is provided. The method includes the steps of: depositing a silicon nitride layer on a silicon on insulator (SOI) starting wafer; patterning the silicon nitride to define at least one silicon microbar; etching the SOI starting wafer to expose the at least one silicon microbar, wherein the at least one microbar is surrounded by a raised perimeter; growing a silicon oxide layer on the raised perimeter of the at least one microbar; and etching a portion of the at least one silicon microbar to produce at least one silicon nanowire adjacent the silicon oxide layer.

In another aspect, a method of detecting a target analyte is provided. The method includes the steps of: providing a silicon nanowire; sensitizing the silicon nanowire with a probe, wherein the probe is specific for a target analyte; obtaining a first electrical measurement from the silicon nanowire; exposing the probe to an unknown solution thought to contain the target analyte; obtaining a second electrical measurement from the silicon nanowire; and determining a change between the second measurement and the first measurement to detect the analyte.

In yet another aspect, a system for detecting a target analyte is provided. The system includes: at least one silicon nanowire, the at least one silicon nanowire having an electrically conductive coating thereon, the electrically conductive coating having a probe that is specific for a target analyte coupled thereto; an electrical measurement system in communication with the at least one silicon nanowire; and a microchannel transverse to the at least one silicon nanowire for introduction of a sample to the at least one silicon nanowire.

Other aspects of the invention will become apparent by consideration of the detailed description and accompanying drawings.

### BRIEF DESCRIPTION OF THE DRAWINGS

FIG. 1 shows a pair of nanowires in cross-section, depicting steps of one method of functionalizing the nanowires with a probe.

FIG. 2 shows a fabrication method for silicon nanowire features. The left side depicts cross-sectional views at the nanowire. The right side depicts top view rotated 90 degrees from the cross-sectional view. Process flow is top to bottom. A). Start with a silicon on insulator wafer. B). Deposit silicon nitride onto the substrate. C). Pattern the silicon nitride and top silicon layers. D). Using a lift off technique, deposit and pattern a Tetramethylammonium Hydroxide (TMAH) etching step. E). Etch out nanowire using TMAH. F). Remove the masking and silicon nitride layers.

FIG. 3 shows a cross-sectional view of silicon on insulator wafer used for the silicon nanowire formation.

FIG. 4 shows a silicon nitride test pattern. The circles both identified the material to be tested, and gave a large enough area for an ellipsometry beam.

FIG. 5 shows a cross-sectional view of silicon nitride deposition step in silicon nanowire fabrication process. A) SOI wafer B) Silicon Nitride deposited.

FIG. 6 shows a histogram of resistance measured with a DC sweep across nanowires fabricated using purely wet chemical etching techniques. The backgate was allowed to float for these measurements.

FIG. 7 shows a histogram of resistance measured with a DC sweep across nanowires fabricated using wet and dry etching techniques. The backgate as allowed to float during these measurements.

FIG. 8 shows poor uniformity silicon nanowire from a combination of wet chemistry and plasma etching techniques. The bright line running top to bottom is the silicon nanowire. The grayish area to the right of the nanowire shows incomplete and non-uniform etching results.

FIG. 9 shows silicon samples after 20 minute phosphoric acid etch. A) Sputtered silicon with no anneal. The circle feature of silicon is no longer visible after the etch. B) Sputtered silicon with anneal. The circle of silicon remains, but some feature definition was lost around the edges C) e-beam silicon no anneal. The circle feature of silicon was completely etched away. D) e-beam silicon with anneal. The silicon circle feature withstood the etch with no loss of feature definition.

FIG. 10 shows silicon nitride wet chemistry patterning. Starting with the silicon nitride coated SOI, photoresist is patterned using photolithography. Silicon is then e-beam deposited over the entire substrate and an acetone liftoff is utilized to remove the unwanted silicon. The sample is then annealed and a phosphoric acid etch is used to remove the unmasked silicon nitride.

FIG. 11 shows a cross-sectional view of substrate during wet etching of SOI and completion of the LOCOS step of fabrication. A). Silicon masked silicon nitride from last step. B). TMAH is used to expose (111) sidewalls in the device silicon. C). Thermal Oxidation is used to grow a passivating silicon oxide on the (111) sidewalls of the device silicon. D). The silicon nitride layer is removed using phosphoric acid.

FIG. 12 shows sputtered germanium after etching. The dark region shows that the germanium remained after the etch and maintained good feature definition.

FIG. 13 shows e-beam germanium after etching (polarizer used to better expose remaining film that couldn't be removed). The lighter region is where the germanium was deposited. The darker region is a surrounding oxide.

FIG. 14 shows backgate etch and nanowire formation steps. A). The silicon microbar structures from the previous process steps. B). A photoresist is applied and patterned to expose a region between the silicon microbars. C). Plasma etching is used to etch through the exposed silicon dioxide to expose the underlying handle silicon. The photoresist is then stripped off using acetone. D). A photoresist is patterned to define the areas of the microbar to become nanowires. E). A masking layer is deposited and the photoresist is removed to liftoff the unwanted material to expose the microbar areas to become nanowire. F). The nanowires are etched out of the microbar structure using TMAH. The mask is then removed.

FIG. 15 shows silicon microbars before metallization and backgates.

FIG. 16 shows current-voltage behavior of silicon contacts on a silicon microbar. This measurement was taken on a microbar applying a −40 μA to 100 μA current across the microbar, allowing the backgate to float.

FIG. 17 shows current-voltage behavior of annealed silicon contacts on a silicon microbar. This measurement was taken on the same microbar as measured in FIG. 16 after an annealing process, applying a −40 μA to 100 μA current across the microbar, allowing the backgate to float.

FIG. 18 shows a silicon standoff and metallization layer. A). Start with the silicon nanowires from the previous process step. B). A photoresist is applied and patterned to expose one contact of the nanowires and the backgate. C), A metal is deposited onto the substrate. D). The photoresist is removed lifting off the metal everywhere except for the backgate contact and a single contact to the nanowires. E). A photoresist is applied and patterned to expose the contact that doesn't have metal on it. F). Another metal is deposited on to the substrate. G). The photoresist is removed lifting off the metal except for in the contact region.

FIG. 19 shows microchannel and device passivation. The microchannel is set up using an ultraviolet definable material, such as SU-8.

FIG. 20 shows a top view of a silicon nanowire array.

FIG. 21 shows silicon nanowire structures.

FIG. 22 shows a close-up view of a silicon nanowire showing good sidewall definition.

FIG. 23 shows an optical image of a completed nanowire chip.

FIG. 24 shows the CVD setup for deposition of DMCS or TMCS deposition onto the sensors. Deposition took place from evaporation of either DMCS or TMCS liquid in the beaker. The tape was placed to mask the contacts from deposition.

FIG. 25 shows microbar sensing of 1 mg/ml BSA-FITc. The red line shows the voltage/current characteristics before BSA exposure, and the green line after exposure. The measurements were taken holding the microbar at a 2 volt potential difference and sweeping the backgate from −20 VDC to 20 VDC.

FIG. 26 shows nanowire sensing of 1 ng/ml BSA-FITc. The blue line shows the current/voltage characteristics before BSA exposure, and the purple line after exposure. The nanowires received a 5 volt potential difference across the wire, and the backgate was swept from −20 VDC to 20 VDC.

FIG. 27 shows a fluorescence image of microbars used for testing. The lighter areas are silicon dioxide, though the silicon did show some fluorescence.

FIG. 28 shows a nickel electroplating setup.

FIG. 29 shows a potentiostat and electrodeposition setup.

FIG. 30 shows a three-probe electrodeposition setup.

FIG. 31 shows a glass slide arrangement in sputter chamber.

FIG. 32 shows an optical image of polymer film (dark area on left half of image) on sputtered silicon. Ellipsometery measurements confirmed a 7 nm polymer film on the silicon region and no deposition on the bare glass.

FIG. 33 shows die contact points for electrodeposition of all microbars/nanowires.

FIG. 34 shows before and after current of silicon microbars. The blue line shows the voltage/current characteristics of the microbar exposed to a −20 to +20 VDC sweep on the backgate and a 5 volt potential difference across the nanowire before electrodeposition of the polymer. The purple line shows the voltage/current characteristics after the electrodeposition took place using the same electrical parameters. The change in the profile is caused by the additional material on the microbar.

FIG. 35 shows test data taken using Raman spectroscopy techniques of a 50 nm polyaniline electrodeposited onto <100> silicon.

FIG. 36 shows Raman data for polyaniline films.

FIG. 37 is a scanning electron microscopic (SEM) image of an exemplary single strand of silicon nanowire.

FIG. 38 is a cross sectional view of the first 4 of 10 process steps used in conventional silicon nanowire fabrication.

FIG. 39 is a cross-sectional view of undesirable SiO₂ growth caused by an insufficient Si₃N₄ diffusion mask used in nanowire fabrication.

FIG. 40 is a SEM image of a single strand of silicon nanowire which was etched completely away due to the lack of integrity of the SiO₂ (111) plane protective sidewalls during a TMAH etch during fabrication.

FIG. 41 shows an ellipsometer measuring an exemplary Si₃N₄ sample.

FIG. 42 is the measured and fitted ellipsometery data from an exemplary Si₃N₄ film.

FIG. 43 is a photograph of an exemplary sample circle array pattern.

FIG. 44 is a graph of the TMAH etch data for an exemplary Si₃N₄ film.

FIG. 45 is a graph showing stoichiometry effects on exemplary Si₃N₄ etch rates.

FIG. 46 shows a diagram of a nanowire sensing system (top) and wiring for electrical measurements from the system (bottom).

FIG. 47 shows nanowire sensing of E. coli.

FIG. 48 shows nanowire sensing of salmonella.

FIG. 49 shows selectivity data for salmonella and E. coli using negative and positive controls.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

In the testing area of biomolecules, there are two dominant approaches currently utilized by both industry and researchers, namely fluorescent tagging and assay detection. Fluorescent tagging is a chemical combination method used for detection of analyte. To use this method, one has to engineer a probe molecule that binds to the analyte of interest in solution. The probe molecule has a fluorescent marker attached to it so that when it binds with the target analyte, it can fluoresce under a specific wavelength.

In this process, a solution with the target analyte is sampled from the media it resided in. This sample is combined with a solution containing the engineered fluorescent probe molecule. After some period of time, which may vary depending on the analyte and probe molecule binding events, the substrate is removed from the solution and rinsed so that only the bound fluorescent probes remain. The substrate is then introduced into a fluorescent microscope to measure the location and concentration of the fluorescent probes to estimate the existence and concentration of the target analyte in the sample.

Fluorescent tagging is a powerful method of detection that has been utilized by a number of industries and researchers for decades. The limits of low concentration detection are being explored by several researchers. In addition, it has been reported that fluorescent tagging has the ability to detect mRNA. With improvements in low-limit detection, this method is still being utilized today. However, there remain significant drawbacks to this label type method, including the fact that it requires substantial time and specialized pieces of laboratory equipment to process and measure samples. In situations where an investigator requires a rapid result in an analyte detection query, the need for lab processing samples and measurements takes too much time and may drive up costs. Other methods need to be developed to solve these problems.

Another method utilized extensively by the medical industry and used throughout the entire market for biomolecular detection is the use of assays that are based on biosensitive devices used for measuring an analyte in solution. Assay detection generally involves a preprocessing step using reactants to help separate the analyte from the solution, an amplification step to decrease the lower limit of detection, and a detection system (e.g. another chemical reaction).

Assays have provided a means of detection for many years. Nonetheless, the pre- and post-processing required to use an assay may have detrimental effects on the reliability of the test. It has been estimated that at least 35% and up to 75% of all medical laboratory assay errors are caused by these processing steps, rather than limitations of the tests themselves. It has also proven very difficult to multiplex an assay-type test, meaning that it would be difficult to integrate this type of test in a determination of multiple analytes during a single test. In the medical field, one of the most frequently used assays it the enzyme-linked immunosorbent assay (ELISA). This test is used for protein diagnostic detection, however it is only sensitive to analyte concentrations down to pico molar (pM) levels. A test that could resolve lower detection limits has the potential to open the door to earlier disease detection.

Nano-scale biosensors have the potential to solve the problems that arise using conventional detection methods described above. Nano-scale biosensors have increased resolution and sensitivity and can typically detect orders of magnitude lower concentration that conventional biosensors due to decreased sensor size. They also have the ability to be packaged into full systems, eliminating the need for pre- and post-processing steps.

Nano-cantilever systems, which are a type of nano-scale sensor, offer ultralow ultimate resolutions, often times in the femtomolar concentration level. Sensing by cantilever usually is done in one of two ways. In both methods, the cantilever has molecules deposited on it that are specifically engineered to bind only with the target analyte. Once this binding event takes place, the measurements are made either by a deflection of the cantilever beam or by a change in the resonance frequency caused by the increased mass attached to the beam. Though this method has proven to be very sensitive, nano-cantilevers are notoriously problematic to fabricate and calibrate. It is extremely difficult to get high yields in mass production of nano-cantilevers due to difficulty in the liftoff step that the fabrication requires. Nano-cantilevers are extremely fragile structures and break very easily both during fabrication and during in use. Though there are very useful aspects of this technology, the problems with the fabrication require that another technology be pursued for mass production of high ultimate sensitivity sensors.

Nanotube materials, whether formed from carbon, boron-nitride or other materials, are another technology that is currently being explored as for possible sensor media. A very common carbon nanotube sensor configuration starts with interdigitated metal electrodes with carbon nanotubes stretching across the gaps between the fingers. The electrodes are patterned using standard ultra-violet lithography and standard deposition and lift-off techniques. A common way of depositing the nanotubes onto the metal is by evaporation of the suspension media in which the nanotubes were purified. This is typically done by dropping the suspension solution onto the area to be deposited, and heating it slightly to increase the rate of evaporation. After the media is evaporated, only the nanotubes remain. The issue with carbon nanotubes as a sensing media is that their electrical properties are orientation-dependent. This is caused by non-uniform band structure throughout the device. The purity of the carbon on the device is also around 95%, meaning that there are other materials that can affect the device sensitivity. The non-repeatability of the device behavior due to the formation of the nanotubes causes this technology to be undesirable for mass production.

Unlike carbon nanotubes, boron nitride nanotubes have electrical properties that are orientation-independent. The deposition method for boron nitride nanotubes is much the same as carbon nanotubes. Though the electrical properties of the nanotubes are better, it is very hard to apply chemical probes to boron nanotubes. Due to the nature of the chemical vapor deposition method required for the formation of boron nitride nanotubes, it is also difficult to make repeatable sensor behavior from one device to the next despite the improvement of feature electronic stability. This technology works well for qualitative sensing, but quantitative sensing requires a method of higher device repeatability.

Silicon nanowires are a technology that offers significant benefits to the lab-on-a-chip platform technology. They have the ability to be more sensitive than current technology because they are physically the same order of magnitude as their analytes that they are sensing. Nanowires by nature have a high surface to volume ratio. In many cases they behave as one-dimensional devices; this allows for a surface interaction to change the electrical properties over an effective cross section of the feature. This changes the electrical properties of the device significantly, ultimately yielding a very sensitive device, having the capability of sub-femtomolar concentration detection in aqueous solution.

Silicon nanowires offer many advantages over other nano-technologies utilized for lab-on-a-chip applications. They are very rugged devices which can withstand much higher mechanical forces than mechanical sensing structures. Silicon nanowires can be produced utilizing CMOS compatible fabrication methods and it is possible to make these devices with standard silicon fabrication techniques.

Silicon nanowire fabrication techniques include the nanoparticle-catalyzed vapor liquid solid (VLS) method, e-beam lithography, and nano-imprint lithography. However, for the present work, a top down process using i-line ultraviolet-based photolithography and anisotropic etching techniques will be used for silicon nanowire formation.

There have been several different methods used to create nanowires which will be briefly discussed here. One of the most heavily utilized methods for making silicon nanowires is the nanoparticle-catalyzed vapor liquid solid (VLS) method. In this method, gold is deposited onto a substrate and then heated to allow the formation of Au—Si alloy droplets on the wafer. These droplets allow for the absorption of the gaseous silicon that is available and the gold-silicon droplet rises as that silicon is absorbed and deposited to harden into a solid underneath the bubble. There are several drawbacks to this method though; the biggest being that the nanowires are not repeatedly reproduced. Though the direction and size can be controlled there are issues with trying to control the orientation and the sidewall roughness that results. Without repeatability it is a less viable option for mass production.

Another common method of silicon nanowire fabrication is e-beam lithography patterned silicon nanowires. In this method, the nanowire pattern is defined by e-beam lithography. Silicon oxide is then deposited in the exposed regions and the rest is then lifted off by removal of the photoresist mask. The nanowires are then etched out using etching techniques. Though this is a good method for making better reproducible nanowires, it takes a lot of time to pattern one wafer and is not currently applicable to the manufacturing scene.

One method that does show some promise of being able to mass produce nanowire devices is a bottom up process relying on nano imprint lithography. This method works a lot like a stamp; a mold is made using e-beam lithography to ensure very sharp and precise features. This mask is typically a flexible material such as PMMA. Once the mask is fabricated, it is removed and a layer of photoresist is applied to the mold. It is then stamped onto the surface of the device substrate to allow for silicon oxide deposition and liftoff as in the process discussed previously. The nanowires are etched out after the liftoff. This process allows for mass production, but nanoimprint processing requires expensive equipment and timely processing to produce the stamping mold to make the imprints with.

The method disclosed herein a top-down fabrication approach for making silicon nanowires. Others have demonstrated a top down fabrication approach using standard microfabrication techniques. In this approach, silicon on oxide (SOI) wafers are used to achieve a two dimensional field on which to pattern a device. With the out-of-plane dimension fixed at a manufactured thickness, oxide is used to mask the regions that are then undercut with TMAH. Rather than using an etch stop method, previous methods use the etching properties of TMAH (the 100 plane etches 100 times faster than the 111 plane) to etch the nanowires out based on time. The known process flow methods yield single silicon nanowires connected to pads. This method yields itself to planarization of the nanowire sidewalls due to the TMAH, however the cross-section of the nanowires produced by these methods is approximately 200 nm in width. However, it is desirable to break below the 100 nm dimension mark in order to maximize the electrical characteristic changes from a binding event. In contrast, the new process paths disclosed herein yields parallel nanowires having much smaller dimensions than achieved by known process paths and achieves these smaller dimensions in a more controllable manner. Accordingly, in various embodiments the methods disclosed herein produce nanowires having a width of less than about 200 nm, less than about 150 nm, less than about 100 nm, less than about 75 nm, less than about 50 nm, or less than about 25 nm.

Accordingly, methods for creating and using a silicon nanowire sensor for rapid, ultra-low concentration bio-chemical sensing applications are disclosed herein, along with models which quantitatively describe the device behavior. A semiclassical model incorporating the physics of the device is disclosed which integrates several key design aspects such as the contact design and the doping concentration of the nanowires that were required for improving the overall sensitivity of the device. Through extensive film characterization studies, the fabrication work disclosed herein identifies a new CMOS-compliant process method utilizing purely wet chemical etching techniques in which to mass produce more repeatable silicon nanowire structures utilizing microfabrication techniques. The probe and analyte work that was performed successfully demonstrate the viability of the silicon nanowire FET device as a high resolution sensing apparatus capable of sensing pico-molar concentrations of target analytes such as BSA-FITc, even before optimization of the probe. The research demonstrated the possibilities to improve the selectivity, sensitivity, and uniformity of nanowire biochemical sensors from what is currently known and/or commercially available.

In one embodiment, the methods and systems disclosed herein include improvements on existing techniques, including techniques for producing silicon nanowires. Improvements on known nanowire formation processes on silicon on insulator (SOI) substrates were achieved by the introduction of a new procedure disclosed herein; an overview of this process is summed up in the following steps:
Silicon nitride is deposited onto the SOI
Ultraviolet lithography is used to pattern silicon microbars on the SOI
The microbars are etched out using anisotropic methods with the silicon nitride remaining on top
Local oxidation of silicon (LOCOS) is performed
An opening is made in the middle of the microbars
Anisotropic etching is used to cut out the nanowires from the microbars.

Through the work disclosed herein, the functional device yield has been increased from 75% to over 95%. One significant advantage of the fabrication paths disclosed herein is that they are CMOS compliant and generally utilize standard process capabilities. Accordingly, the disclosed processes are expected to be compatible with many semiconductor fabrication facilities while remaining low in cost.

The disclosed methods of fabrication allow for the mass fabrication of silicon nanowire arrays. These methods allow for a few different applications including silicon nanowire sensor devices. These have applications in industrial, medical, and research applications.

In certain embodiments, the fabrication is based off of i-line lithography technology, local oxidation of silicon (LOCOS), and anisotropic etching of crystalline silicon. One particular embodiment of the process flow is laid out in a step by step basis as follows:

1. A <100> plane silicon on insulator (SOI) wafer is chosen as the starting medium. The properties of the SOI wafer are <100> 650 micron thick silicon handle wafer (the bottom part of the wafer), with a 145 nm thermally grown silicon oxide layer (the middle of the wafer) and a 70 nm <100> silicon device layer (top of the wafer). The oxide of the wafer allows for a thick enough dielectric to provide electrical passivation between the device silicon and the handle silicon, yet thin enough to allow for gating effects between the two layers by allowing for alteration of transport properties, which is very important to the functionality and control of the intended device being fabricated.

2. The SOI wafer is cleaned using an RCA clean. This process utilizes Ammonium Hydroxide and Hydrogen Peroxide to remove the organics on the wafer, Hydrofluoric Acid to remove the oxide formed by the organic removing solution, and then a mixture of Hydrochloric Acid and Hydrogen Peroxide to remove the ionic contaminants of the wafer. This cleaning process is standard for prefurnace wafer cleaning because it ensures that there are no contaminates on the wafer prior to deposition.

3. The next step is the start of the modified LOCOS process. A 100 nm Silicon Nitride is deposited on the wafer using low pressure chemical vapor deposition (LPCVD), a process where heated gasses are combined under a vacuum to deposit the desired film on a batch of substrates. After a lot of testing on different methods of deposition of the silicon nitride on the wafer, it was found that LPCVD was the most desired method because it formed the highest density silicon nitride and the most repeatable stoichiometric film. It also allows for several wafers to be deposited at once versus one at a time. The high density of the silicon nitride film is important for the LOCOS process because it allows the film to act as an effective oxygen diffusion barrier. As found with other methods of deposition, the less dense films allow oxide growth on the silicon below the silicon nitride and also form an oxynitride layer which greatly complicates the process flow. The stoichiometry of the silicon nitride films is important because it allows for repeatable etching processes in boiling phosphoric acid, which will be explained in greater detail further down the process.

4. A patterned photoresist is made using i-line photolithography. The mask was designed to allow the open areas to be the dogbone like shapes and the rest of substrate be masked.

5. An e-beam deposition of silicon to 35 nm takes place. The thin layer of silicon is used as a masking layer for the phosphoric acid etch in the next few steps.

6. An acetone ultrasonic bath is used to lift off the silicon on top of the photoresist from step 4. This works by chemically dissolving the photoresist leaving the undesired silicon floating in solution. In order to make the silicon denser to survive the acid dip, the sample is annealed in a nitrogen ambient at 1000 C for one hour.

7. A boiling phosphoric acid dip is performed to remove the unprotected silicon nitride. The Acid is held at 85% concentration diluted with deionized water and boils at 165 C. The etch takes place for 30 min.

8. A short 5 second dip in 10:1 deionized water to 49% Hydrofluoric acid (10:1 HF) removes the unwanted silicon oxide on the exposed device silicon. The samples are dipped in 25% Tetramethylammonium hydroxide (TMAH) diluted with water heated at 65 degrees C. for two minutes. This is an anisotropic etchant that etches the 100 plane of silicon 25 times faster than the 111 plane. This step serves multiple purposes: it etches the unprotected device silicon, it removes the silicon layer on top of the silicon nitride, and it smooths out the sidewalls of the device silicon under the silicon nitride. It is important to note that the device silicon under the silicon nitride is at 54.47 degrees which will allow for an etch stop to be formed in the next step.

9. The sample is put in a dry oxidation furnace to grow a thermal oxide on the exposed silicon. The silicon nitride that is left covering the dogbones acts a diffusion barrier for the oxygen allowing for the LOCOS process to take place. This process takes place at 950 C for 15 min in an oxygen ambient.

10. Following the oxidation, the sample is dipped for 6 seconds in 10:1 HF. This etches the small layer of oxynitride that results from the dry oxidation and allows for the rest of the remaining silicon nitride to be removed using another boiling phosphoric acid dip.

11. A photolithography step is done to pattern a photoresist to allow for the back gate connection. This pattern leaves an opening for reactive ion etching (RIE) to burn through the silicon oxide.

12. Using Carbon Tetrafluoride (CF4), the exposed silicon oxide is removed using RIE. This allows for a connection to the handle wafer to be made from the front side of the wafer.

13. The masking photoresist is stripped using acetone. The sample is then lithographically patterned again for the lift off of the masking layer for the silicon nanowires to be etched out. This mask leaves a small area over the center of dogbones exposed to allow for only that region to become parallel nanowires.

14. Germanium is the sputtered onto the sample to act as a masking layer for the TMAH etch that has to happen to make the nanowires out of the dogbones. It was found that sputtered Germanium stood up better than E-beam evaporated germanium to the TMAH etch.

15. The sample is then put into an ultrasonic acetone bath to lift off the unwanted germanium on the substrate. This leaves germanium on everything except for the small opening where the silicon nanowires are going to be formed.

16. The germanium is then annealed for 15 minutes at a temperature of 450 degrees C. in a nitrogen ambient.

17. The sample is dipped in 10:1 HF for 5 seconds to remove any native oxide on the device silicon and then dipped into 25% TMAH for two minutes. The sample is then rinsed and dipped into CR7 for one minute to remove the germanium without adversely affecting the remaining silicon, or the silicon oxide.

18. The sample is then patterned using photolithography to make possible a liftoff for the metallization layer. The open areas of the photoresist after this step are where the metal is going to remain.

19. Electron beam evaporated aluminum is then deposited on the substrate to a thickness of 35 nm.

20. The sample is then put into an ultrasonic acetone bath to lift off the unwanted aluminum from the substrate.

A first alternate process flow embodiment uses other materials in place of germanium (e.g. chromium) in steps 14-17. However, one drawback to this embodiment is that it takes away the CMOS compliancy of the process flow. Accordingly, a second alternate process flow embodiment uses RIE for the pre-patterning of the LOCOS steps. It is an alteration of the first few steps of the process flow:

1. The SOI is cleaned as described above.

2. The silicon nitride is deposited using LPCVD.

3. The wafer is then patterned using photolithography. The photoresist covers the dogbone features this time instead of leaving them exposed like the last process flow.

4. The exposed regions of silicon nitride are then etched away using CF4 and RIE.

5. The photoresist is then stripped away using acetone, and the sample is dipped in 10:1 HF for 5 seconds to remove the native oxide from the exposed silicon. The sample is then dipped in TMAH for 2 minutes. The rest of the process from here on forward with the backgate, the metallization and the formation of the nanowires proceeds as explained above.

In other embodiments, the order of the formation of the backgate, the metallization and the nanowire formation steps can be changed. This would still yield the same device, however the path first described has been found to be the highest yielding path while still maintaining CMOS compliancy.

Accordingly, in one embodiment a method for fabricating silicon nanowires includes the steps of: providing a silicon on insulator (SOI) starting wafer, the SOI wafer including a handle wafer base, a silicon oxide layer on the handle wafer, and a silicon device layer on the silicon oxide layer; depositing a layer of silicon nitride on the silicon device layer of the SOI starting wafer using low pressure chemical vapor deposition; applying a patterned photoresist to the silicon nitride layer, leaving a plurality of open areas lacking the patterned photoresist; depositing a silicon layer on the patterned photoresist and the silicon nitride layer using e-beam deposition, wherein portions of the silicon layer that are directly applied to the silicon nitride layer act to protect the silicon nitride layer and wherein the remaining portions of the silicon nitride layer are unprotected; removing the patterned photoresist from the SOI starting wafer; removing the unprotected portions of the silicon nitride layer; exposing the SOI starting wafer to etchant to remove the portions of the silicon layer protecting the silicon nitride; growing a thermal oxide on the exposed silicon; applying a photoresist mask over the plurality of open areas; sputtering a protective metal over the SOI starting wafer; removing the photoresist mask over the plurality of open areas; etching the plurality of open areas to generate a plurality of silicon nanowires; and depositing aluminum on the silicon nanowires using electron beam evaporation.

In another embodiment, a method of detecting an analyte includes the steps of: providing an aluminum-coated nanowire made using the methods disclosed herein; performing a first measurement of an electrical property of the aluminum-coated nanowire; contacting the aluminum-coated nanowire with the analyte; performing a second measurement of the electrical property of the aluminum-coated nanowire; and determining a change between the second measurement and the first measurement to detect the analyte.

Overview of Analyte Detection Methods and Systems

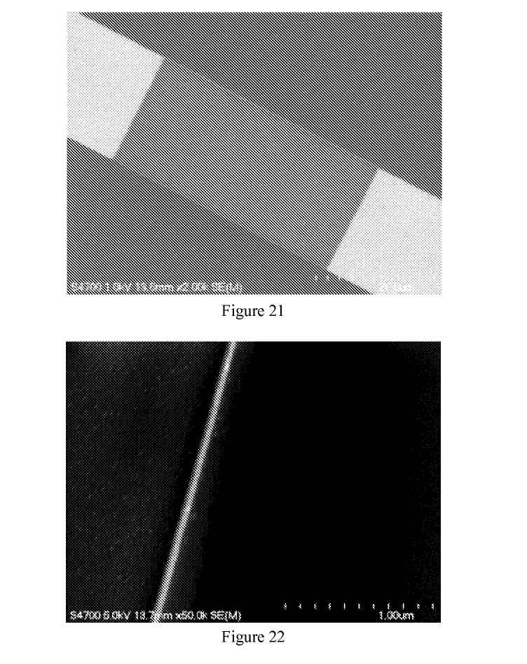

The present disclosure provides methods and systems for detecting one or more analytes. As an initial step, one or more silicon nanowires is produced using the methods disclosed herein. In certain embodiments, one pair of nanowires may be produced for each microbar on a substrate, as shown in FIGS. 20 and 21. Groups of microbars may be formed adjacent to one another on a substrate to produce an array of nanowires on the substrate.

The pairs of nanowires may be used together as a unit, e.g. the same probe may be applied to both and measurements may be taken from both, or one nanowire may be isolated, e.g. by breaking the connection of the second nanowire of the pair, so that electrical measurements are taken from only one nanowire of the pair. In some embodiments, the pairs of nanowires may each have different lengths, as shown in FIG. 20. The different length nanowires have different sensitivity levels to the target analyte and as such a given length nanowire may be better suited to a particular concentration range of target analyte.

Next, the nanowires are functionalized to sensitize them to a target analyte of interest. Nanowires may be functionalized by attaching a probe specific for the target analyte to each of the nanowires.

One way to functionalize the nanowires is to apply a conductive coating to the nanowires and to subsequently attach the probe to the conductive coating. Possible conductive coatings include metals or polymers such as electrically conductive conjugated polymers. Possible metals include aluminum, iron, titanium, and nickel. Polymers include polyaniline, polyacetylene, poly(p-phenylene vinylene), polyfluorene, polyindole, polycarbazole, polyazepine, polypyrene, and polyacetylene. Important factors for selection of a material include the ability to perform electrochemical deposition with good adhesion to silicon and electrically conductivity of the applied material, to allow for charge transfer between the probe molecules and the nanowire sensor.

The coating may be applied using electrochemical deposition and/or by selective masking. With either technique, one can apply different probes to different nanowires in order to produce an array of nanowires which together can sense a variety of target analytes.

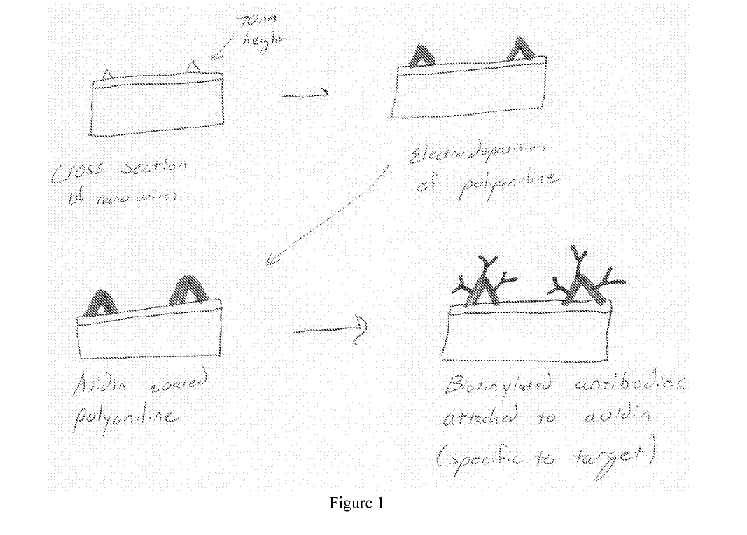

Probes may be coupled to the nanowire coating using covalent or non-covalent interactions, or combinations thereof. For example, FIG. 1 shows a pair of nanowires in cross-section which are coated with polyaniline by electrochemical deposition. The polyaniline is in turn coated with avidin and biotinylated antibodies are then attached to the avidin (due to the strong interaction between avidin and biotin). Thus, the sensor shown in FIG. 1 is specific for the target analyte that is recognized by the antibodies.

Using the approach shown in FIG. 1, nanowires can be selectively coated with polyaniline in a stepwise manner. After each polyaniline coating step, the subsequent steps required to attach a specific probe (e.g. an antibody as in FIG. 1, or other probe) are completed. Only those nanowire(s) that are coated with polyaniline (or other coating) will be functionalized with the particular probe in that step. In subsequent steps, other nanowires may be coated with different probes using a similar approach, to produce an array of nanowires with sensitivity to a variety of target analytes.

Other techniques that may be used to couple probes to nanowires include hydrozone bonding (which also relies on the electrodeposited polyaniline layer) using hydrazide and aldehyde, or covalent techniques such as silanization of silicon.

In general, the localized deposition of a material onto one location to the exclusion of others usually requires some ability to enhance the binding energy for the deposition to one material over others. This can be done by electrochemical methods, as disclosed herein, for example by polymerizing a monomer on site or by electrochemical reaction of a salt to form a solid from the solution (i.e. as in electroplating a metal), or by other chemical deposition means. One example of other means includes the localized chemical vapor deposition of tungsten metal locally into a semiconductor contact, where the tungsten deposition conditions can be defined so that nucleation does not occur on the oxide regions surrounding the contact, but only inside the contact and only on top of the semi-metal diffusion barrier material. This sort of localized chemical vapor deposition has been demonstrated extensively at higher temperatures (>400° C.) and in submicron layer thicknesses. Localized chemical vapor deposition may be extended to low temperatures and nanoscaled layer thicknesses using metal-organic precursor materials and the atomic layer deposition process, which allows for monolayer by monolayer growth of a material from a precursor chemical that can decompose into a solid inorganic or cross linked into some organic framework.

Thermal enhancement of binding is another possible method. As the nanowire is made of a different material than the surrounding surfaces (Si vs. SiO₂ or Si₃N₄, or other conductive vs. insulative material), a light source or other source of heat may be used to cause a higher localized temperature on the nanowire layer relative to the dielectric. Thus, the binding energy would be enhanced on the wire compared to the dielectric and the deposition could be localized to that spot.

Other methods include the generation of photo-induced or electron- or ion beam-induced binding locally on the nanowire by scanning the nanowire with a laser in an appropriate source vapor ambient, or in an environmental scanning electron microscope or a focused ion beam system with a chemical vapor source tube localized near the electron or ion beam source.

As mentioned above, another possible deposition method is selective masking. A sacrificial layer may be applied to the substrate (or may be present from an earlier fabrication step) and may be used later in the processing sequence to 1) deposit the coating over the whole surface of the sample, and 2) remove the unwanted area of deposition by the removal of the sacrificial layer from beneath the bound coating. A related method is to use the same or a secondary mask which is patterned over the nanowire area to expose that area and protect the remaining portions of the sensor platform.

Yet another method of functionalization of nano-semiconductor features utilizes a thin oxidation layer followed by OH binding of probe molecule to that oxide layer.

Probes may include proteins such as antibodies and nucleotides such as DNA or RNA, any of which has been designed or selected to find to a target analyte with a high degree of specificity. Target analytes include any material that can be detected in an aqueous solution, including bacteria, viruses, fungi, cells/cell markers, inorganic chemicals, organic chemicals, proteins, and nucleic acids. Specific targets include Salmonella, Listeria, Norovirus, mi-RNA, E. coli, coliform bacteria, chlorine, nitrogen, phosphorous, ebola, pharmaceuticals, chemical warfare agents, industrial chemicals, radiological byproducts, and effluent products of pharmaceuticals and chemical products.

The coated and functionalized nanowire array sensor may be coupled to a microfluidic system for delivery of materials to the sensor array and for removing spent samples. To facilitate this, one or more microchannel may be formed on the substrate which crosses the nanowire sensors transverse (including perpendicular) to the nanowires.

Electrical properties of the nanowires are monitored in order to sense changes in the nanowires which arise from binding of the target analyte(s) to the probe(s) associated with one or more nanowires. In some embodiments, an oscillating voltage is driven across the nanowires and subsequent changes in the conductance or impedance of the nanowires is monitored before, during, and/or after exposure of an unknown solution (which may contain the target analyte) to the nanowire or nanowire array. In various embodiments, other electrical measurements that may be used to monitor changes in the nanowires include capacitive or frequency domain relationships.

In use, a substrate having one or more nanowire thereon is functionalized as described above so that the nanowire(s) on the substrate contain one or more probes directed to one or more target analytes. An unknown sample which may contain some of the target analytes is applied to the nanowire(s), for example using a microfluidic delivery system coupled to a microchannel which directs fluid across the nanowire(s). One or more electrical properties of the nanowire(s) is measured before, during, and/or after exposure of the nanowire(s) to the sample. Changes in electrical properties are then used to determine whether one or more target analytes are present and the concentration of the analytes. Electrical measurements can be converted to concentrations by comparing electrical values obtained from different known concentrations of the target analyte in test solutions.

Device Design

Described herein is a model of the behavior of the silicon nanowire sensors which shows theoretical electrical properties of the sensors. Modeling to describe the contact behavior and nanowire behavior changes from binding events and backgating biases is also disclosed. Experimentation was also carried out to confirm the modeling results.

The contact behavior of the nanowire sensor devices is described. From this work, it was found that making ohmic contacts to the lightly doped silicon nanowires was possible, although inconsistent from one device to the next. These results led to an exploration into Schottky contacts and their effect on the device. It was found in this study that it was possible to fabricate a single directional device by choosing the metallization materials for both contacts to allow for no potential barrier in the same direction. These results were confirmed experimentally. Modeling was also performed to find the theoretical sensitivity of the wires and the gating effects of the nanowire. This work advanced the understanding of the silicon nanowire sensor device by enhancing the understanding of the contacts and confirming the sensitivity of the nanowires and backgating effects.

Silicon Nanowire Sensor Platform Fabrication

Also described herein are methods of implementing a top down fabrication path for repeatable production of devices at high yields. Extensive work went into the exploration of the fabrication of silicon nanowires. Among other improvements, the fabrication path for the production of the nanowires was altered from known methodologies to incorporate etch stops on every step to improve the uniformity and manufacturability of the technology. Individual studies were performed on every step of the new process path to characterize the film and chemical interactions to maximize the functional yield of the substrates.

Several features of the improved process path contribute to the overall repeatability of the fabrication process for top the down silicon nanowire sensor fabrication disclosed herein. The silicon nitride film must be dense enough to mask oxygen diffusion while remaining easy to remove; the silicon nanowires have improved features when prepared using purely wet chemical etching methods; and masking materials need to be carefully selected so as to not interfere with the surrounding exposed layers. Using techniques as disclosed herein, the overall functional yield was slightly above 95%, whereas the known process path only yields only 75%.

Probe and Analyte

Also disclosed herein are studies showing that the nanowire-based devices operate as sensing devices. The probe/analyte interaction and effects on the nanowire were explored in this work. BSA-FITc was initially used as a target analyte because it is a readily available molecule that can be identified via fluorescence to confirm its presence on the sensor independent of the electrical measurements. Measurements down to pico-molar concentrations were tested and found to be detectable with the nanowires. Other selective probe coatings such as nickel and polyaniline were explored and deposition using electrochemistry techniques was shown to be feasible. Both of these films have future applications ranging from blood testing to water quality sensing, and the electrodeposition methods disclosed herein show that it is possible to selectively coat the nanowires with these materials.

Silicon Nanowire Sensor Design and Function

The design of the nanowire features and the contacts is important for achieving high sensitivity; for example, separating the contacting electrodes from the sensing region allows for electrical isolation between the two pads. This design prevents the nanowire sensor from shorting out while operating in a conductive aqueous solution. In order for the device to behave properly, the nanowires themselves need to be electrically isolated from the aqueous solution as well. In many cases the resistance of the nanowire features is so high that the majority of aqueous solutions that the sensor is exposed to will provide a path of conduction of less resistance. In other cases, to reduce or eliminate the possibility of shorting out the sensing feature of the device, the nanowire may be coated in an insulating material or the probe molecules may be used which have insulating properties to them.

The silicon nanowire sensors used in this work are chemo-electrical sensors. The silicon nanowires may be fabricated in arrays and may have one or more microchannels running transverse (including perpendicular) to the nanowires, and analyte probes may be attached to the nanowires. In general these probes are engineered to chemically bind to a particular target analyte with a high level of specificity. Accordingly, when a target analyte is bound to the probe, the binding changes the surface charge properties along the nanowire. Due to the large surface to volume ratio of the nanowires, the changes in surface properties (which may be relatively small) result in significant changes in the electrical properties of the nanowire which can be measured, for example by driving a signal through the system and measuring properties such as impedance changes. The binding effects can be further enhanced if the target analyte has charge-modulating properties. It has been shown that relatively little charge transfer takes place in the presence of native oxide on the nanowires; this means that it is likely that the changes in the electrical properties of the nanowires is caused by Coulomb interactions. In addition, both N-type and P-type nanowires have shown to be effective in sensing applications.

Physics Behind Nanowire Devices

The general model that is utilized for the function of nanowire sensors is of a semiclassical MOSFET device. The nanowires act as the channel of conduction, controlled by the gate bias. In the case of a nanowire device, the control gate electrode can be the handle substrate, or a top gate. This model works well for many of the parameters of the device behavior, however, it does not fully explain other features which should be modeled to fully understand the device behavior.

It has been shown that the lighter the doping on the silicon nanowires, the more sensitive the device is to the target analyte and that smaller devices may have higher sensitivity. However, there are still many aspects of the binding events that take place during sensing that affect the nanowires in ways which are not understood.

The metallization contact behavior and how it affects the nanowires has not been fully explored. Some published work describes utilizing different contact methods such as silicides, metal sintering, and implant doping. The contact design has the ability to fully deplete the nanowire, or partially deplete it based on the work functions difference of the two materials at the junction interface and the size of the interface. By proper design, the selections of the work functions can be used as an advantage to enhance the ultimate resolution of the sensor.

Another gap of knowledge is how the surface roughness produced during fabrication of the nanowire channel impacts the overall sensitivity of the device. The surface roughness can be minimized by adjusting process parameters; however, it cannot be completely eliminated. Surface imperfections cause trap sites and other defects in the device; these have the potential to lower the sensitivity of the device and reduce the ultimate resolution.

The impact of impulse response or frequency response of the nanowire system has not been characterized. The use of impedance measurements combined with the biasing capabilities of the sensor creates a need for broad exploration of operational possibilities for the nanowires that warrants further investigation. Biasing of the nanowires plays a huge role in the overall electrical properties of the nanowire: with relatively small voltages, it is possible to fully deplete the device, or accumulate charge within the device. Impulse response and frequency response have been all but ignored by other researchers up to this point. The natural frequency of the nanowire structures will be significantly altered by the change in charge density when a probe attaches to the target analyte. Frequency response could potentially be much more sensitive than a measurement of the conductance change. Impulse response may also have similar benefits.

To produce a satisfactory commercial embodiment of a silicon nanowire device will require addressing technical drawbacks to further the understanding of nanowire device physics, and creating a repeatable mass production fabrication process for nanowire sensors will allow for the overcoming of the barriers currently blocking this device from being commercialized. Given that the nominal resistance of the nanowires is 3 GΩ, it is important to have consistency and repeatability between devices. In one test, the measured current change for detecting a pico-molar concentration of target analyte was 100 nA; in view of such high nanowire resistance and small currents of detection, unrepeatable nanowires with different resistances would make for unreliable results between sensors. High yield is also extremely important in the commercialization of silicon nanowires because semiconductor processing is expensive, and producing more functioning devices decreases per device costs. The methods disclosed herein will resolve many of the above-identified issues to facilitate production of a full functioning commercial device.

The following non-limiting Examples are intended to be purely illustrative, and show specific experiments that were carried out in accordance with embodiments of the invention.

### EXAMPLES

### Example 1

### Silicon Nanowire Sensor Platform Fabrication

This Example details the fabrication work that was performed to create a process capable of producing uniform nanowire sensors in high yields and high volumes. Known process paths for silicon on insulator silicon nanowire sensors have a number of limitations, which the disclosed methods improve over.

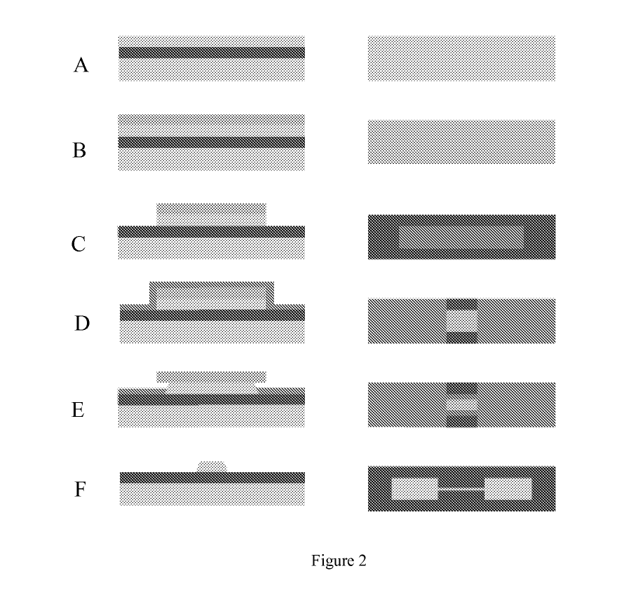

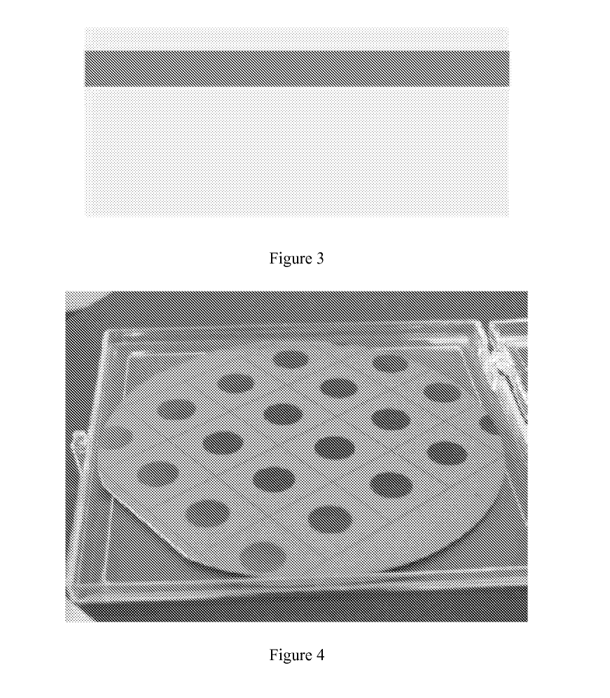

A top-down process method was selected as the most viable candidate for high volume production and ease of integration into existing semiconductor fabrication facilities. A modified version of the methods of Stern et al. (E. Stern, R. Wagner, F. J. Sigworth, R. Breaker, T. M. Fahmy, M. A. Reed, “Importance of the Debye Screening Length of Nanowire Field Effect Transistor Sensors,” Nano Lett, Vol 11, pp. 3405-3409, November 2007) was used for the fabrication of silicon nanowire sensors because it had the most advantages when compared to other nanowire fabrication methods. FIG. 2 shows the nanowire formation portion of the process flow; the left images show the cross-sectional view of the nanowire area, and the right images depict a top-down view. Starting with a (100) plane SOI wafer (FIG. 3), silicon nitride was deposited (FIG. 5) using low pressure chemical vapor deposition (LPCVD). The silicon nitride's purpose in this process was to act as a masking layer during anisotropic etching of the top silicon of the SOI wafer referred to as the device silicon. Using ultraviolet lithography, a microbar was patterned and then etched out of both the silicon nitride and the device silicon. Another lithography step was performed to define the silicon nanowire length from the microbar, and a chemical masking layer was deposited and lifted off. The exposed faces of the device silicon under the silicon nitride were then chemically etched using an anisotropic etchant to reduce the feature size of the microbar into a nanowire of desired feature size based off of time of the etch. Once this step was completed, the masking layer was removed and metallization was performed to set up contacts to the silicon.

This fabrication process has a number of desirable characteristics. The ability to pattern nano-features using micro fabrication technology enables high volume manufacturing of the sensors at lower costs than would be possible using standard nanofabrication techniques. Another distinct advantage to this process path is the lack of complexity of the mask set required. Not only was the required resolution of the dimensions of the mask set low in comparison to nanofabrication techniques, the number of masks required for the formation of the nanowire structures was only two: one for the microbars and one to define the length of the nanowires.

There were a few prominent disadvantages to the process path of Stern et al. for silicon nanowires that has led to unsuccessful integration into scaling the process up. The first issue that needed to be addressed in the process flow is the contact design. With the device dimensions at the nanometer scale, the contact effects were amplified when compared to their larger counterparts. Modeling work performed has identified design solutions to this problem which are implemented into the fabrication path. The most problematic fabrication issue that arose from this process path was the lack of repeatable etching of the nanowire: the methods of Stern et al. do not include etch-stops in the process path. The size of the nanowire was determined by a multitude of variables, including but not limited to time, chemical strength and variability in chemical composition, doping concentration, and temperature. The variations that arose from this method were outside of acceptable limits for the readout electronic design and therefore a better implementation of etch-stop techniques was required. The final issue that was addressed was that of wire surface roughness and etching techniques that were used to mitigate some of that roughness.

Beginning of Revised Process Silicon Nanowire Process Path

Disclosed herein are process steps for the fabrication of CMOS-compliant silicon nanowires sensor chips. The silicon nanowire fabrication work was carried out using 8″ Soitec SOI wafers. The device silicon (top layer) was 10 Ω/cm p-type, <100> orientation, and was 70 nm thick. The oxide was 145 nm thick, and the handle wafer was 10 Ω/cm p-type, <100> orientation and 650 m thick.

The start of the fabrication sequence was to cleave the substrate into quarters to reduce the substrate size to a dimension that would allow for processing at Michigan Technological University's Microfabrication Facility. Subsequent to cleaving the substrates, an RCA clean was performed. This clean is a CMOS-compliant process that removes contaminants from the substrate using an organic bath, a hydrofluoric acid bath, and an ionic bath. The procedure calls for full immersion baths making use of the following chemistries: 1:1:8 ammonium hydroxide:hydrogen peroxide:deionized water heated to 90° C. for ten minutes, followed by a 30 second room temperature 100:1 hydrofluoric acid:deionized water bath, followed by a 10 minute 1:1:9 hydrochloric acid:hydrogen peroxide:deionized water dip heated to 90° C. Between each bath step and after the final bath the samples were put into rinse cycle tanks using deionized water. The samples were dried individually using 99.999% nitrogen.

Silicon Nitride Deposition

After the RCA clean was performed, the next step called for the deposition of silicon nitride onto the device silicon of the SOI substrate. Historically, silicon nitride has been a vital film in silicon microfabrication. The local oxidation of silicon (LOCOS) method has many applications including MOSFET fabrication. This film is utilized in the LOCOS process because it is a dielectric that can withstand high temperatures and effectively mask oxygen diffusion during device passivation steps. Not only is the diffusion of oxygen through this material slow, but stoichiometric silicon nitride does not oxidize quickly, allowing for an easy means of removal if desired. Due to these film properties, silicon nitride plays a very important role in the beginning steps of the formation of silicon nanowires as well. In the final process path for fabrication of silicon nanowire sensors presented in this work, the silicon nitride layer must perform three tasks:
The silicon nitride must be thick enough to mask the device silicon layer from oxygen diffusion during a 15 minute 950° C. dry oxidation.
The silicon nitride must be able to withstand 25% tetramethylammonium hydroxide in deionized water at 65° C. for 4 minutes.
The silicon nitride must be easily removed after the required masking steps are completed.

There are many different ways to deposit silicon nitride, including low pressure chemical vapor deposition (LPCVD), radio-frequency (RF) sputtering, e-beam physical deposition, and reactive RF sputtering. A study was conducted to identify the most advantageous method of deposition for the nanowire fabrication. In various embodiments of this process flow, the silicon nitride films preferably exhibit the three characteristics listed above.

The RF sputtered samples were deposited using a 99.99% purity stoichiometric silicon nitride target in a Perkin-Elmer Randex sputtering system model 2400. The ultimate pressure prior to deposition was 2.2×⁻⁷ torr, and operating deposition pressure was 2.2×10⁻² torr. In RF sputtering, a compound material such as silicon nitride does not deposit on the substrate with the same stoichiometry as the target. This is due to differences in the diffusion of the elements out of the target during the ion bombardment process described above, and recombination of elements within the process chamber prior to deposition onto the substrate. To find the proper combination of nitrogen and argon gas chemistries that would allow for nearly stoichiometric silicon nitride to be deposited, the gas ratios were varied as shown in Table 1. The films were deposited on 1″ by 1″ silicon pieces and measured using ellipsometric techniques.

TABLE 1
RF SPUTTERED SAMPLES FABRICATED WITH
DIFFERENT GAS RATIOS

| | N2:Ar Ratio | Stoichiometry |
|---|---|---|
| Sample 1 | 0:1 | 29.7% Si rich |
| Sample 2 | 3:200 | 1.9% Si rich |
| Sample 3 | 1:19 | 2% N rich |
| Sample 4 | 3:17 | 7.9% N rich |
| Sample 5 | 4:16 | 9% N rich |

The reactive RF sputtered silicon nitride was deposited using a 99.99% purity silicon target in a Perkin-Elmer Randex sputtering system model 2400. The ultimate pressure was 2.1×10⁻⁷ torr, and deposition pressure was 1×10⁻² torr. The gas ratios used were varied as shown in Table 2 to define different possible stoichiometries that would be produced. The stoichiometries in this table are averages for multiple samples.

TABLE 2
REACTIVE RF SPUTTERED SAMPLES FABRICATED
WITH DIFFERENT GAS RATIOS

| | N2:Ar Ratio | Stoichiometry |
|---|---|---|
| Sample 1 | 0:1 | 100% Si rich |
| Sample 2 | 1:3 | 48.9% Si rich |
| Sample 3 | 1:1 | 42.3% Si rich |
| Sample 4 | 3:1 | 36.7% Si rich |
| Sample 5 | 1:0 | 4.39% N rich |

Silicon nitride was e-beam deposited using a Denton DV-502A. The material used was 99.9% purity stoichiometric silicon nitride chunks in a graphite crucible. The ultimate pressure was 7.5×10⁻⁸ torr. Consecutive runs using the same material led to an evaporation material stoichiometry change due to the difference in evaporation rates of silicon and nitrogen. This led to wildly inconsistent stoichiometries to be deposited onto the substrates, ultimately ruling this form of deposition out for the final process flow.

The LPCVD stoichiometric and low-stress silicon nitride films were deposited using a Semy LPCVD stack capable of processing 6″ wafers. The stoichiometric silicon nitride was deposited using 25 sccm dichlorosilane and 75 sccm ammonia at 800° C. The low-stress silicon nitride was deposited using 75 sccm dichlorosilane and 25 sccm ammonia at 800° C. The tube was heated at a slight ramp, from 790° C. to 810° C. from back to front in order to make the deposition more uniform across the boat by changing the reactivity of the gasses as they are spent during the reaction process.

The samples for this study were fabricated using 4″ 10 Ω/cm p-type <100> prime silicon substrates. The samples were RCA cleaned and then put into the respective tools for silicon nitride deposition. After the deposition was completed, the samples were lithographically patterned, and the silicon nitride films were etched using CF₄ plasma. The remaining photoresist was then stripped to yield wafers with the pattern in FIG. 4. These wafers were then cleaved into individual dies, and the resulting samples were used for testing.

To make the most accurate comparison of the investigated film deposition methods and their resulting film qualities, the three desired characteristics of the silicon nitride film for this process flow were tested independently from one another. All film measurements were made by a J. A. Woollam VVASE 400 ellipsometer, and the stoichiometry measurements from this tool were confirmed using a Hitachi S-4700 FESEM with x-ray detection capability.

Utilizing an effective medium approximation (EMA) layer, the stoichiometry of the silicon nitride films could be measured. The EMA layer also allowed for more accurate measurements of the silicon nitride film thickness because it could account for variations in the properties of the silicon nitride. In more detail, the EMA layer provided with the WVASE32 software is used to characterize a film in which one material is suspended within another material. This technique is not meant to measure metal alloys, but it is designed to measure impurities and overall elemental make-up of a compound material. This layer works best when well defined materials are used. In this case, stoichiometric silicon nitride was the main material, and the silicon content of the film was manipulated to measure the presence or absence of silicon content within the silicon nitride film. Throughout this work, the Bruggeman approximation was used for the blending of optical properties for the EMA layer.

Unless otherwise stated, the measurements were taken from 300 nm to 1000 nm in 10 nm increments and from 65 degrees to 75 degrees in 5 degree increments. The experimental data was then modeled using a 500 μm crystalline silicon layer, a thin silicon dioxide layer for the native oxide (between 1-2 nm), and an EMA layer, in that order, to measure the silicon nitride. The EMA layer consisted of stoichiometric silicon nitride and decoupled silicon to show the addition or subtraction of silicon from stoichiometric silicon nitride. The thickness of the silicon dioxide, the silicon nitride layer, and the silicon content of the silicon nitride were all allowed to be variables during the model fitting process, which utilizes algorithms to automatically fit the model parameters to the measured data.

The goal of the first test was to define the minimum thickness for the silicon nitride film to be an effective oxygen diffusion barrier. All samples had stoichiometric silicon nitride deposited except for the low stress LPCVD silicon nitride. The LPCVD samples were patterned as shown in FIG. 4. They were then cleaved and etched in 165° C. phosphoric acid to yield thicknesses in 4 nm intervals. Measurements were taken with the ellipsometer to confirm the targeted thickness. The sputtered silicon nitride films were deposited off center to the target to create a gradient in deposited film thickness. The samples were then patterned as shown in FIG. 4. Utilizing that gradient allowed for testing of several different thicknesses. After the preparation of the silicon nitride spots on the wafers, the samples were then oxidized using dry oxygen at 950° C. for 15 minutes. After the wafers cooled down, they were etched in 165° C. phosphoric acid to remove the silicon nitride. Measurements were then taken to see if any silicon dioxide growth took place under the masking layer. Table 3 shows the critical cutoff thicknesses for effective oxygen diffusion masking.

TABLE 3
EFFECTIVE OXYGEN DIFFUSION MASKING THICKNESSES

| Si₃N₄ Deposition | Minimum Film |
|---|---|
| Method | Thickness |
| CVD Stoichiometric | 94 nm |
| CVD Low Stress | 96 nm |
| Sputtered | 138 nm |
| Reactive Sputtered | 132 nm |

The second experiment for the silicon nitride layer was to test the silicon nitride film's ability to mask against 65° C. TMAH. The samples were produced in the same manner as described in the previous experiment. To make the data more complete, the extremes of the stoichiometry were used as well as stoichiometric silicon nitride from the sputtering tools. After fabrication of the silicon nitride circles, the samples were cleaved and put into TMAH one at a time. The samples were removed and measured at 2 minute intervals using the ellipsometer to determine the etch rate. Each sample underwent 20 minutes of time in the TMAH, much longer than what is actually required by the final silicon nanowire process. Table 4 shows the results from this study.

TABLE 4
SILICON NITRIDE ETCH RATES IN TMAH

| Si₃N₄ Film and | |
|---|---|
| Deposition Method | TMAH Etch Rate |
| CVD Stoichiometric | 0 nm/min |
| CVD Low Stress | 0 nm/min |
| Sputtered 30% Si Rich | 0 nm/min |
| Sputtered Stoichiometric | 0 nm/min |
| Sputtered 9% N Rich | 0 nm/min |
| Reactive Sputtered 36.7% Si Rich | 0 nm/min |
| Reactive Sputtered Stoichiometric | 0 nm/min |
| Reactive Sputtered 4% N Rich | 0 nm/min |

The final silicon nitride study was to test the etch rate in 165° C. boiling phosphoric acid. The temperature was held constant by controlling the water content of the phosphoric acid rather than by controlling the temperature of the solution. The samples were fabricated using gas ratios described above in Table 1 and Table 2. The samples were placed into the phosphoric acid bath and removed at two minute intervals to measure the etch rates. The numbers were averaged between samples from the same deposition type and on samples to give the average etch rate for the film deposition method. These results are shown in Table 5.

TABLE 5
SILICON NITRIDE DEPOSITION METHODS AND
THEIR PHOSPHORIC ACID ETCH RATES

| Si₃N₄ Film and | |
|---|---|
| Deposition Method | H₃PO₄ Etch Rate |
| CVD Stoichiometric | 4.5 nm/min |
| CVD Low Stress | 2.3 nm/min |
| Sputtered 30% Si Rich | 5.5 nm/min |
| Sputtered Stoichiometric | 11.9 nm/min |
| Sputtered 9% N Rich | 21.7 nm/min |
| Reactive Sputtered 36.7% Si Rich | 4.3 nm/min |
| Reactive Sputtered Stoichiometric | 11.2 nm/min |
| Reactive Sputtered 4% N Rich | 20.5 nm/min |

The silicon nitride study was conducted to determine the best deposition method for the silicon nanowire fabrication process. For this, many aspects needed to be taken into account, and it was concluded that for single wafer processing, reactive RF sputtering is the best deposition method. Nevertheless, to form a process flow that can be easily scalable into high volume manufacturing, the preferred choice given these test results is stoichiometric LPCVD silicon nitride.

Etching Techniques and their Impact on Nanowire Uniformity

The next step in the process path was to pattern the silicon nitride to begin the LOCOS process. At this point, it was necessary to designate whether purely chemical etching methods or a combination of wet and dry etching methods produced better uniformity and device structures. A study was conducted to determine which etching method utilized throughout the fabrication process would yield the most desirable nanowire features.

It has been determined that lower surface roughness along the nanowire yields more uniform behavior, leading to a more repeatable sensor. Accordingly, the present study was conducted to determine which fabrication methods led to the least amount of surface roughness along the <111> plane of silicon, along with the most uniform and repeatable nanowire structures. The samples for this study were silicon nanowires fabricated using the methods described above, with variations on the etching techniques. One set of samples utilized a fabrication process incorporating purely wet etching techniques, and the other set of samples utilized a combination of wet and dry etching techniques. The testing of the repeatability of the nanowire fabrication and the surface roughness were indirectly measured using impedance measurements from a Keithley 2400 semiconductor parameter analyzer.

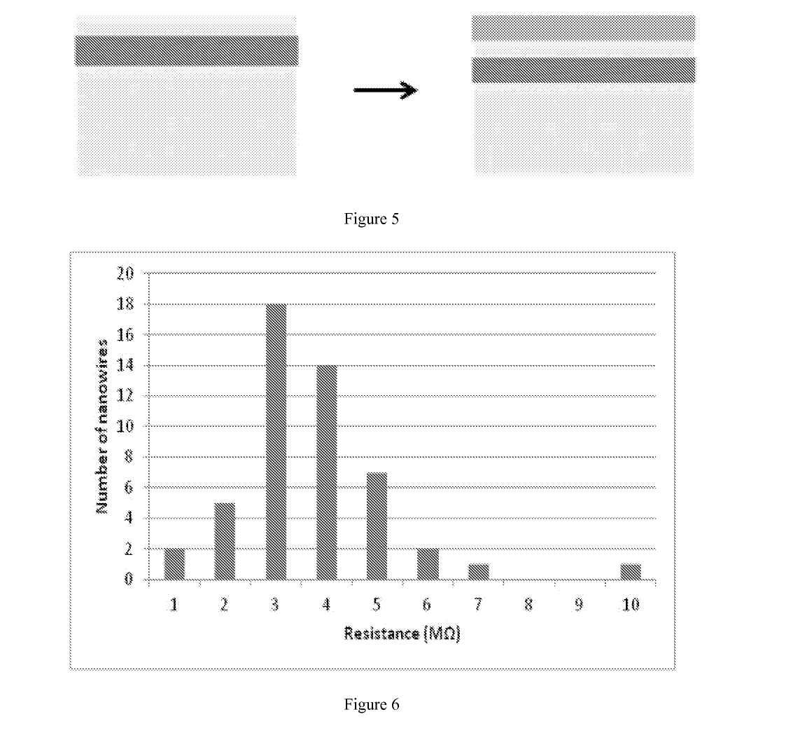

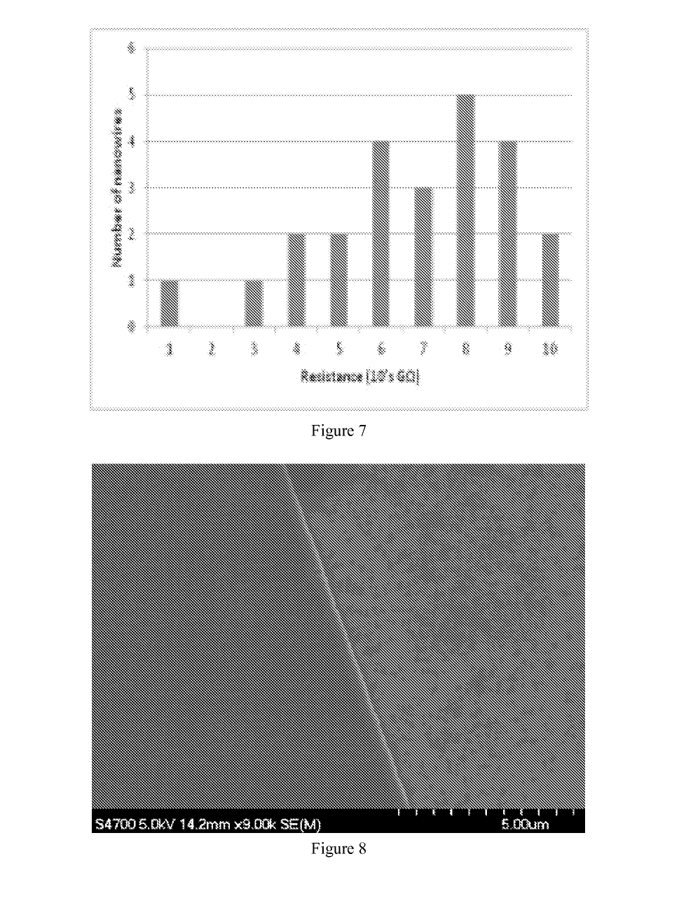

Approximately 75 samples using the different etching techniques were fabricated. The results of the measurements made on these samples are shown in the histograms depicted in FIG. 6 and FIG. 7. Different length features were used in these histograms between the purely wet chemistry etching and combination of wet and dry etching. This was done to save processing time and substrates. The measurements were performed on devices in different locations on a wafer and across different wafers.

The distributions of the two histograms in FIGS. 6 and 7 are significantly different: the wet chemical etching method has a standard distribution of 1.52 MΩ while the wet and dry etching method had a standard distribution of 22.84 MΩ. Accounting for the difference in length, the purely wet chemical etching fabrication method still yields more uniform wires with a tighter (smaller) standard deviation. The overall functional device yields were 75% and 95% for wet/dry and wet etching fabrication, respectively.

The results above indicate that a purely wet chemical etching fabrication approach produced the highest yield and the most uniform nanowire features. Through this study, it was determined that there are at least two reasons that this is the case. Chemical etching techniques allow for much higher selectivity of etching materials, allowing for longer and more controlled etching. In nanowire fabrication, this allowed for the silicon nitride to be etched more effectively than with plasma etching because the selectivity of the wet chemistry etching method was 5 times higher between silicon nitride and silicon than the dry etching method. This added selectivity permitted an etch time that might otherwise produce over-etching for this silicon nitride to allow for it to be completely removed. Due to the nature of plasma etching, the device silicon on the outer parameter of the wafer ended up thinner than the device silicon in the center of the wafer. Also the dry plasma attacked the silicon oxide passivation layer on the nanowire 10 times faster than the wet chemistry etch did. Ultimately, the selectivity enhancement of the wet chemistry allowed for more controlled etching of the nanowire resulting in more uniform results.

Selectivity aside, the nature of plasma etching also creates non uniformities in device structures. As explained above, the method of film removal by plasma etching is both a chemical and physical process, which can be modeled using Monte Carlo simulation. The randomness of the ion bombardment of the sample creates the possibility for uneven etching, and has the potential to slightly skew a patterned device in the nanoscale range. This may lead to nanowires that are not straight lines. The combination of uneven etching and poor uniformity of the nanowire that results from these issues is shown in FIG. 8.

Silicon Nitride Masking Layer Study

In the finalized process flow for the fabrication of silicon nanowires there are two chemical masking layers that are used. One layer protects the silicon nitride from 165° C. phosphoric acid and the other layer protects the device silicon from 65° C. TMAH. The masking layer for the silicon nitride etch will be discussed here because it is the next part of the fabrication sequence; the masking for the TMAH step will be discussed further below. There were several possible masking materials that could be used in this situation, and this study was performed to determine which material would be best suited for the job. To reduce the number of materials for experimentation, all metals were excluded in order to remain CMOS compliant with the fabrication process flow. Table 6 shows the two chemicals that need to be masked, and the possible materials that can be effectively used. These materials were chosen because they are CMOS compliant and they can be deposited on multiple wafers simultaneously using chemical vapor deposition methods to satisfy the scalability requirements. Table 7 shows the etching chemistries for the possible masking materials and their effects on other exposed materials.

TABLE 6
POSSIBLE MASKING MATERIALS AND THEIR ETCH RATES

| | | 90% H₃PO₄ at |
|---|---|---|
| | 25% TMAH at | 165° C. Etch |
| Material | 65° C. Etch Rate | Rate^{[45]} |
| Poly silicon | 103 nm/sec | .7 nm/min |
| Silicon dioxide | 0 nm/sec | .18 nm/min |
| Silicon nitride | 0 nm/sec | 4.5 nm/min |
| (stoichiometric | | |
| LPCVD) | | |
| Poly germanium | >200 nm/sec | .13 nm/min |
| Poly silicon | >150 nm/sec | 4 nm/min |
| germanium | | |

TABLE 7
POSSIBLE MASKING MATERIALS AND THEIR ETCH RATES

| | Masking | Masking | Silicon | Silicon | (100)Crystalline |
|---|---|---|---|---|---|
| Etching | Material | Material Etch | Nitride Etch | Dioxide Etch | Silicon Etch |
| Chemistry | Etched | Rate | Rate | Rate | Rate |
| 25% TMAH at 65° C. | Poly silicon | 103 nm/min | 0 nm/min | 0 nm/min | 300 nm/min |
| 1:10 HF:H₂O at | Silicon dioxide | 23 nm/min | 1.1 nm/min | 23 nm/min | <1 nm/min |
| room temp | | | | | |
| 90% H₃PO₄ at | Silicon nitride | 4.5 nm/min | 4.5 nm/min | .18 nm/min | 17 nm/min |
| 165° C. | (stoichiometric | | | | |
| | LPCVD) | | | | |
| CR7 Chrome | Poly | 260 nm/min | <1 nm/min | <.1 nm/min | 0 nm/min |
| etchant | germanium | | | | |
| 126:60:5 | Poly silicon | 550 nm/min | 0 nm/min | 8.7 nm/min | 150 nm/min |
| HNO₃:H₂O:NH₄ | germanium | | | | |
| at room temp | | | | | |

After the material selection portion of the study was completed, it was determined that poly silicon is a preferred material selection for the masking layer for the silicon nitride etch. Poly silicon can withstand the chemistry and the temperatures of hot phosphoric acid etching better than germanium, the another possible alternative. Not only can poly silicon mask the silicon nitride effectively, but the subsequent step to the silicon nitride etch is an anisotropic silicon etch. The chemistry used for this step can remove the poly silicon masking layer while etching the device silicon, eliminating the need for an additional etching step.

Following the completion of the material selection portion of the masking study, an additional study was conducted to determine the best available deposition methods and annealing parameters available for the silicon mask. The deposition methods that were explored were RF sputter deposition and e-beam deposition. Various annealing parameters were used following the deposition step.

Ellipsometry was used to measure the film characteristics during this study. The general oscillator layer provided with the WVASE32 software was used to model the amorphous silicon and polycrystalline silicon layers deposited using both e-beam and sputtering, respectively. This layer describes optical properties of materials based on oscillation functions that are controlled by wavelength, or photon energy. In the case of silicon, the Tauc-Lorentz model was used.

The substrates used for this study were 4″ 10 Ω-cm p-type <100> silicon wafers. The samples were subjected to an RCA clean to remove contaminants and then were loaded into a furnace for dry thermal oxidation. The oxide was needed to accurately measure the poly silicon film thickness using ellipsometry. After the oxidation, the substrates were patterned for lift off, loaded into the silicon deposition tools, and received a film. After the film was deposited, the remaining photoresist was removed using acetone ultrasonics, yielding a pattern similar to FIG. 4. The substrates were then cleaved into single structure samples and annealed at various temperatures in a nitrogen ambient.

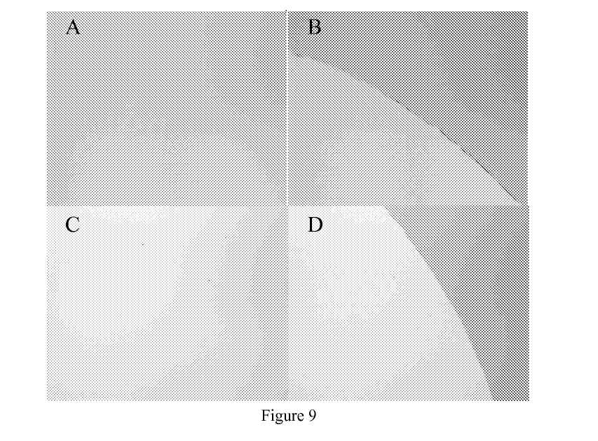

After the fabrication and annealing of the test samples was completed, the samples were subjected to 165° C. phosphoric acid in two minute intervals. The remaining silicon thickness was measured at two minute intervals using the ellipsometer to verify the etch rates. The results are shown in FIG. 9. From these results, it was clear that e-beam deposited silicon and a 1000° C. anneal for 1 hour in a nitrogen ambient yielded the highest quality masking layer. Without the annealing step, the silicon was etched away by the phosphoric acid.

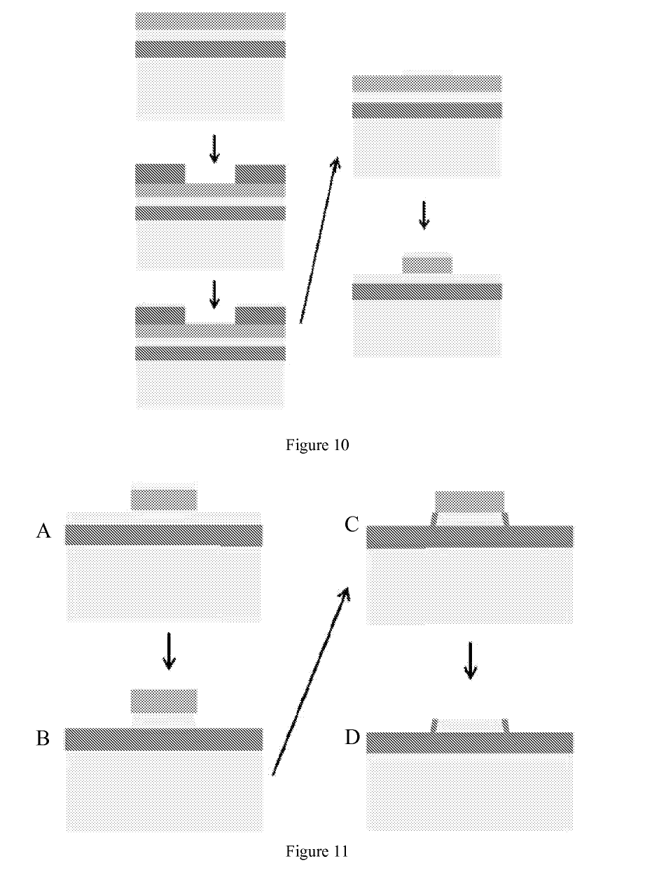

The results from the masking study were included into the fabrication as shown in FIG. 10. The recently deposited silicon nitride was patterned using i-line photolithography. Following the lithography step, a 35 nm silicon film was deposited using e-beam deposition. The remaining photoresist mask was removed using acetone ultrasonics, leaving just the desired masking regions of the silicon. This silicon was then annealed at 1000° C. anneal for 1 hour in a nitrogen ambient to make the film poly-crystalline in order to increase its chemical resilience against phosphoric acid. The sample was then put in a 165° C. phosphoric acid bath for 30 minutes to remove the 100 nm silicon nitride film.

Completion of LOCOS Process

After the patterning of the silicon nitride was completed, both the exposed device silicon and the poly silicon mask are etched using a 4 minute 65° C. TMAH etch. The remaining silicon nitride was used as a masking layer to leave behind the microbar structures after the exposed device silicon was etched to the (111) plane. The fabrication process continued as shown in FIG. 11. The masking silicon layer and the device layer were etched in TMAH at 65° C. for 4 minutes. Though the etch time was much longer than what was actually required to etch through 70 nm of (100) plane crystalline silicon and the poly silicon mask, the extended time was found to give smoother sidewalls from the etching techniques study. Following the formation of the microbars and the removal of the poly silicon mask, a dry oxidation was performed. This grew the protective oxide layer on the <111> planes shown in FIG. 11. The silicon dioxide acted as an etch stop during the nanowire etch later in the fabrication process. Following the oxidation, the silicon nitride was removed using a phosphoric acid bath at 165° C. for 35 minutes. Wet chemistry was chosen as the method of film removal because it had better film selectivity than dry etching methods, and allowed for the removal of silicon nitride with the least amount of damage to the final device. The film selectivity for the CF₄/O₂ plasma etching was about 6:1 silicon nitride to silicon dioxide. The film selectivity for the 165° C. phosphoric acid etch was nearly 50:1 silicon nitride to silicon dioxide; this created an etch stop that allowed for better control than the plasma etching making a preferred choice for the process path.

Backgate Contact Opening and Nanowire Etching

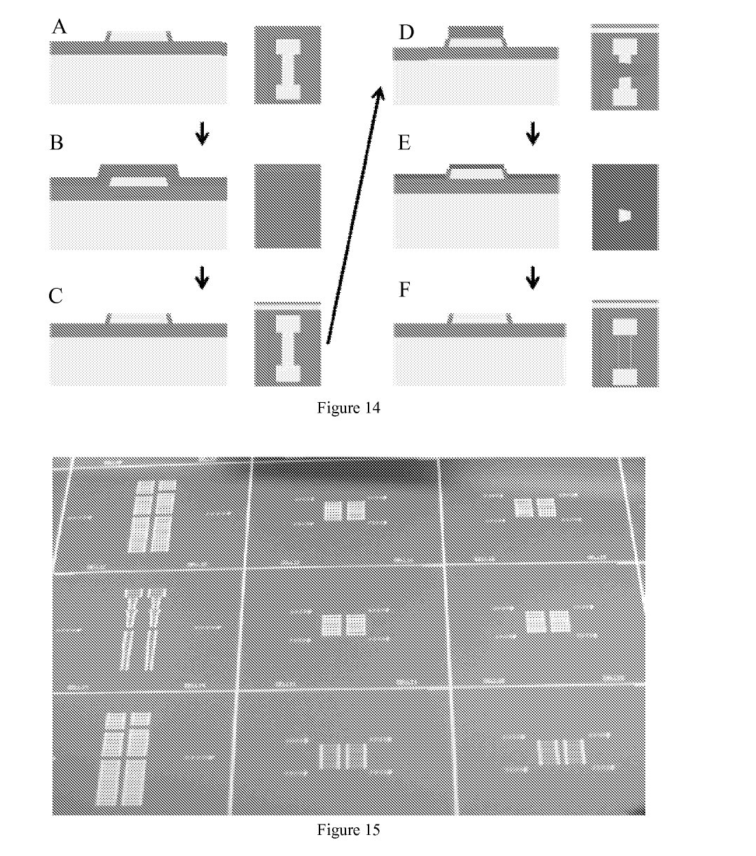

The next portion of the fabrication that was performed was opening the backgate connection to the handle wafer and etching out the nanowires from the microbars. As shown in FIG. 14, the backgate opening was lithographically patterned and etched out using CF₄ plasma. CF₄ plasma was chosen because it was the fastest and most reliable method to etch only the contact opening; over etching was not an issue because the handle substrate is 650 m thick, and the sidewall roughness was not a factor. The selectivity between silicon dioxide and the photoresist was substantially higher with the CF₄ plasma etch than a buffered hydrofluoric or similar wet chemical etch.

From the results of the etching techniques study, it was concluded that purely wet chemistry etching methods yielded the highest uniformity nanowires with the smoothest sidewalls. As a result, a masking material needed to be chosen for the TMAH etch to form the nanowires. Table 6 and Table 7 from the silicon nitride masking layer study reveal that several materials can be used to mask TMAH, however there is only one material of the possible masking materials listed in these tables for which the etchant has the selectivity needed to leave the other exposed films unaffected. That was the film chosen for the masking layer, germanium, and an etchant that removes germanium cleanly, CR7.

Germanium Mask Layer Study

As stated in the above section, germanium was chosen as a masking layer for the etching of the microbar silicon into nanowires. It was chosen because germanium can mask TMAH and can be removed without harming the surrounding layers of the sensor device. There exist many ways that germanium can be deposited, and the characteristics of the resulting films needed to be investigated.

The methods of deposition that were investigated were e-beam deposition and RF sputtering. The substrates used for this study were 4″ 10 Ω-cm p-type <100> silicon wafers. The samples were subjected to an RCA clean to remove contaminants and were then patterned for lift off using photo lithography. Following the lithography step, the samples were loaded into the deposition tools and received a 35 nm germanium film. After the film was deposited, the remaining photoresist was removed using acetone ultrasonication and yielded a pattern similar to FIG. 4. The substrates were then cleaved into individual devices for etch testing.

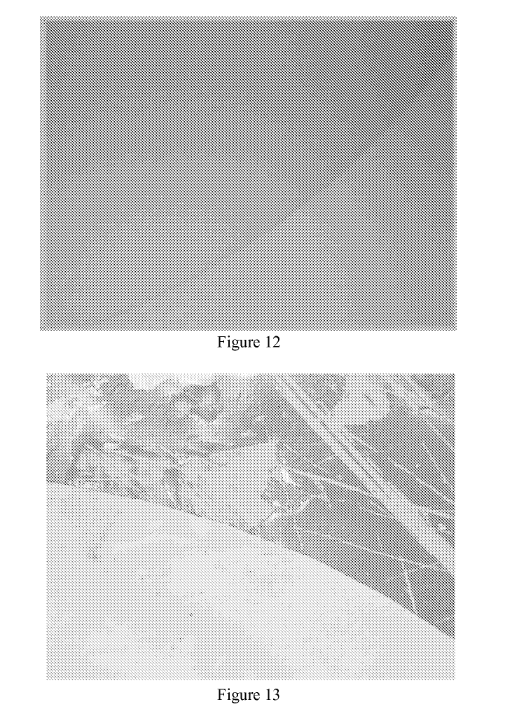

After the fabrication of the test samples was completed, the samples were subjected to 65° C. TMAH in 2 minute intervals. The samples were measured at 2 minute intervals to verify the etch rates. The results are shown in Table 8, and FIGS. 12 and 13. All of the films effectively masked the TMAH; however, the sputtered film was most cleanly removed using CR7. These results allowed for the conclusion that sputtering was a preferred method for germanium masking deposition.

TABLE 8
TABLE 3.8. GERMANIUM MASKING ABILITY

| Ge Deposition Method | TMAH Etch Rate |
|---|---|
| Sputtered | 0 nm |
| e-beam | 0 nm |

Upon completion of the study, the microbar received a photoresist patterned for liftoff to allow for application of the germanium masking layer. The germanium was sputter deposited to a thickness of 35 nm, and the photoresist was removed using acetone ultrasonication. The nanowires were then etched out of the microbars using a 2 minute 65° C. TMAH etch. The nanowire etch was completed by removing the germanium with room temperature CR7 for 2 minutes.

Silicon Annealing Study

After the formation of the nanowires, the next portion of the fabrication of the sensor platform was the metallization. The metal selection and depletion region setup play a role in the functionality of the device. Adding a heavily-doped silicon layer between the lightly-doped substrate and the metal contact helps the functionality of the device by pulling the depletion region set up by the semiconductor-metal interface out of the nanowire sensing area. One portion of this design that needed to be tested was the effect of the crystalline state of the silicon on the contact behavior of the device. This study was performed to investigate how the crystalline state of the contact silicon affects the transport of the complete device.

The samples for this experiment were silicon microbars fabricated in the same methods described above without the nanowire etching. This yielded a structure as shown in FIG. 15 below. From this point, lithography was used to define photoresist for the lift off process for the contact silicon to the device. The contact silicon used in this study was 10¹⁸ boron doped 99.9999% purity silicon with 0.0015 Ω-cm resistivity. After the liftoff pattern was defined, the contact silicon is deposited to 120 nm using e-beam deposition. Following the deposition, the unwanted silicon was removed using acetone ultrasonication.

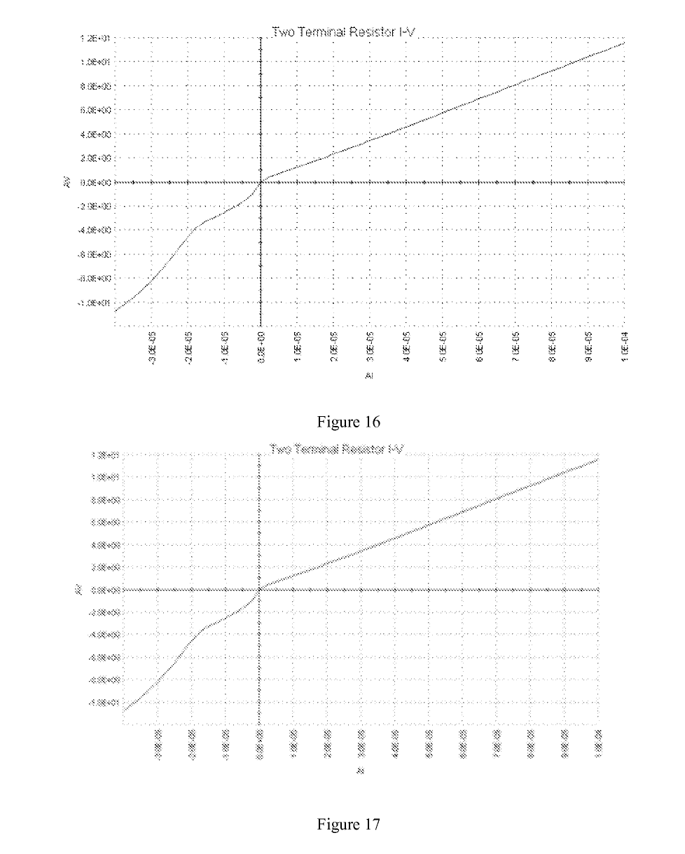

After the substrate reached this point, the individual sensor dies were cleaved out of the substrate. Some samples were annealed in a nitrogen ambient at 900° C. for 20 minutes, and the remaining samples did not receive the anneal. The experiment used 5 devices of both annealed and unannealed silicon to allow for a sufficient number of devices to be tested to confirm the results. Following this stage, the samples received the same photolithography mask used for the contact silicon liftoff, and glass microscope slides were strategically placed during the metal deposition process to mask the sample. This temporary masking effectively allowed for only one side of the microbars to have metal deposited at a time. Metal was sputter deposited onto the device, the slides removed, and a different metal was deposited on the other side. This set up a 1-directional device as described herein. The current vs. voltage was measured using a Keithley 4200 semiconducting parametric analyzer (SPA). The results are shown in FIG. 16 and FIG. 17 below.

As seen by comparing FIG. 16 and FIG. 17 above, annealing has almost no effect on the measured current. The resistance of the unannealed device was slightly higher than the annealed device. Without being limited by theory, it may be that the non-ideal waver in device current characteristic from the modeling disclosed herein for this device is caused in part by the change of majority carrier type at the interfaces. This study concluded that annealing the contact silicon is not required for the desirable behavior and repeatability of the overall device, however further investigation into the behavior of the device may be warranted.

Metallization

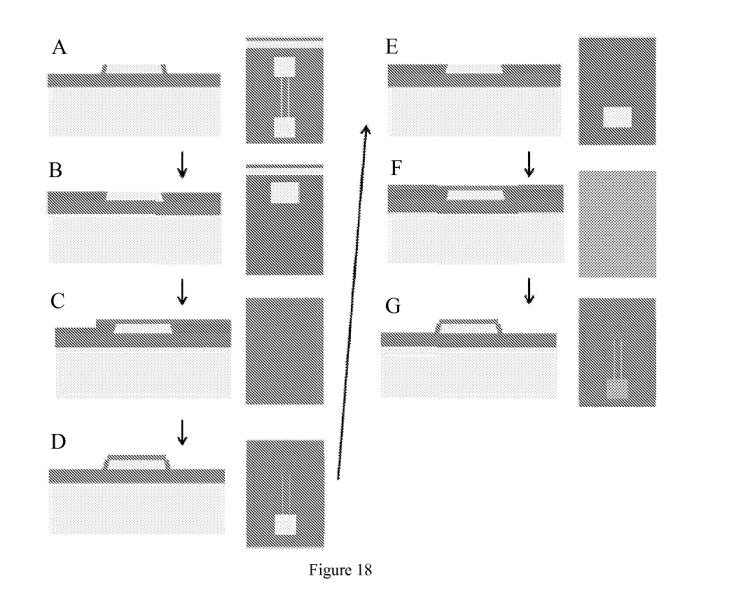

FIG. 18 depicts the metallization process steps. Starting from the nanowires, a photoresist was patterned for liftoff, leaving only the backgate and one side of the contacts open. The p++ contact silicon described in the previous study was e-beam deposited onto the substrate, and acetone ultrasonication was used to lift off the unwanted material. The substrate then received a 900° C. anneal in a nitrogen ambient for 20 minutes. Following this, another photoresist was patterned for lift off to expose the backgate and one of the contacts. A metal was then deposited using e-beam deposition. The photoresist was then lifted off using acetone ultrasonication, and another photoresist was applied and patterned to expose only the remaining contact. A different metal was deposited and the photoresist was removed using acetone ultrasonication.

Micro Channel and Passivation

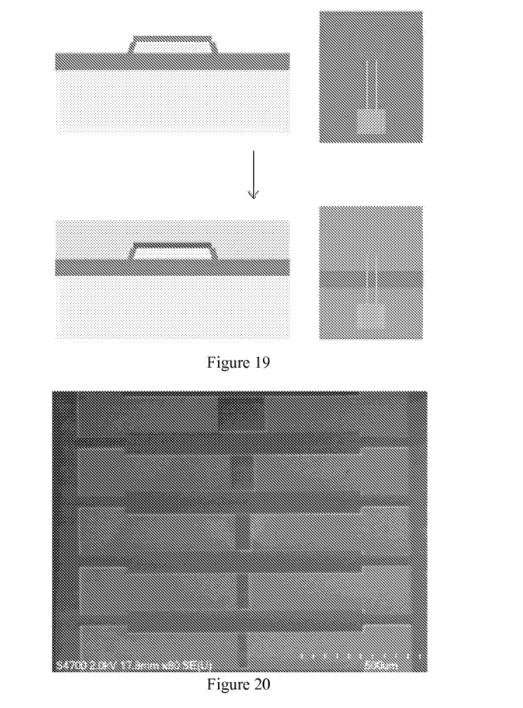

Following the completion of the nanowire sensor platform, a micro channel needed to be fabricated on the substrate to allow for the solution to be measured to travel only on the sensing regions of the sensor. For this, a simple photo lithography step with SU-8 was used. This allowed for a 50 m high and 100 m wide. The process is shown in FIG. 19. This step concluded the sensor fabrication.

Results

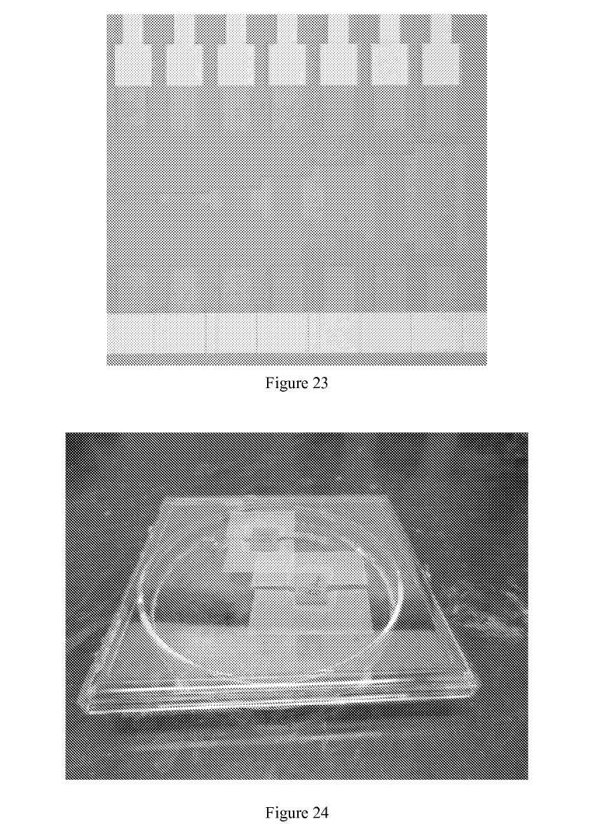

Upon the completion of the studies discussed above and characterization work for the individual process steps, the overall functional device yield was improved from 75% to 95%. FIG. 20 shows one of the arrays fabricated from this work. FIG. 21 shows one of the resulting nanowire structures from the microbars. The nanowires are located on the outsides of the structure. The difference in gray tones between the nanowires and the exterior of the nanowires is due to different silicon dioxide thicknesses due to processing. FIG. 22 shows an FESEM image of one of the nanowires from the finalized process flow. This image shows that the nanowires have very well defined smooth sidewalls and uniform shape. Finally, FIG. 23 is an optical image of one of the arrays showing the completed passivation and microchannel.

Summary and Scalability of Process

Accordingly, a number of processing experiments were performed to identify and characterize a preferred process flow for repeatable production of silicon nanowire sensors. The process presented here has a number of advantages over known process paths, including the process path presented by Stern et al. One addition to this process flow was to include etch stops in the form of passivated silicon nanowire sidewalls. Doing so eliminates a number of process variables that would have made it difficult to obtain repeatable devices. The preferred fabrication process also utilizes purely wet chemical etching methods to produce the nanowires, reducing the sidewall roughness and improving overall device function.

Although some minor changes may be needed to make the flow scalable for high volume manufacturing, it is expected that the general flow will remain the same. One area where changes may need to be made is in the semiconductor masking layers. Rather than rely on a serial deposition processing procedure such as e-beam deposition, LPCVD can be utilized for the poly silicon and poly germanium masking layers. However, the patterns would have to be etched in rather than being lifted off, as in the present work. This would mean that the patterning steps would take place after a blanket film deposition; with the underlying materials that are exposed during these steps, this is not expected to present a problem with etching unwanted materials.

The process path presented here utilizes purely wet chemical etching techniques for the formation of the nanowires. This also allows for batch processing, which works well in a manufacturing scenario. Overall, a complete and repeatable process flow for silicon nanowire fabrication was found which yielded over 95% functional devices.

### Example 2

### Probe Materials and Deposition Methods

This Example details the experimental work which was performed to transform the microbar and nanowire devices fabricated according to the methods disclosed herein in order to test probe-analyte combinations to show sensing capability.

As described above, the silicon nanowire sensor platform functions through chemical interactions which in turn change the electrical characteristics of the nanowires by changing the surface energy. The probe molecules used to bind to the target analyte were chosen or engineered to selectively bind to only a given analyte of interest in a solution containing an unknown sample. When the probe molecule binds with a target analyte from the solution, the surface properties of the nanowire are changed. These changes of the nanowire can be measured by modulation in impedance, which was monitored by driving an electrical signal through the wires. A variety of signal properties were considered, a DC current, an AC current, and a variety of waveforms to test the differences in sensitivity that could result. The output was continuously monitored; the output changed as the targeted analytes bound to the nanowire surface.

Several experiments were performed to show that the presently-disclosed silicon nanowires have the ability to function as a sensing apparatus. Ideally, a probe molecule should only be bound to the sensing region of the sensor device. However, this has proven to be a difficult aspect to achieve with confidence. A first set of tests involved using Bovine Serum Albumin (BSA) which incorporated a fluorescent tag. Following this study, several selective materials were investigated.

The first analyte tested with this sensor was fluorescein isothiocyanate-labeled bovine serum albumin (BSA-FITc). BSA-FITc was chosen because the binding events that took place between the probe applied to the sensor and the BSA-FITc in solution behaved very similarly to other analytes of interest. In addition, the fluorescein isothiocyanate (FITc) label allows for fluorescent imaging of the bound BSA to identify the molecule following an incubation period of the samples. Finally, BSA-FITc was selected because it is an inexpensive test molecule that is widely used for proof of concept of biosensing applications. There exists a broad range of literature on BSA-FITc binding behavior. This broad background combined with availability of the molecule allowed for extensive testing to be performed at minimal cost to the project.

The linking probes chosen for this experimentation were dimethylchlorosilane (DMCS), and trimethylchlorosilane (TMCS). The process complexity to use these two molecules was minimal, again allowing for several experiments to be conducted with minimal complexity.

There were several aspects of the probe binding to silicon and the analyte that required investigation. As a result there was a wide array of samples used during this study. The sample type and fabrication will be given in detail for each step described herein.

The first goal of the BSA-FITc binding study was to demonstrate binding of the probes to silicon. During the time of application, there were two materials that the DMCS and TMCS would be exposed to, namely silicon and silicon oxide.

For one iteration of this study, four samples of 1″×1″ cleaved 10 Ω-cm p-type <100> silicon substrates were fabricated. Two of the samples were oxidized by exposure to molecular oxygen gas at 950° C. for twenty minutes. These two samples were produced for the testing of the binding ability of DMCS and TMCS to silicon oxide. The bare silicon samples were exposed to a 10:1 mixture of deionized water to hydrofluoric acid by volume for 30 seconds to remove the native oxide. After rinsing the samples, all four substrates were immediately mounted to two sample cases. The configuration utilized was one bare silicon sample and one oxidized sample per sample case. Each individual sample case was then put over a beaker filled with either DMCS or TMCS for 30 minutes as shown in FIG. 24. This allowed for a chemical vapor deposition method to deposit either DMCS or TMCS on the exposed substrates. After the completion of the deposition process, the samples were rinsed in deionized water.

Following the completion of the fabrication process for the substrate and probe molecules, they were exposed to BSA solution. The samples were dipped into 1 mg/mol concentration BSA solution for 30 minutes. The samples were then removed and placed in phosphate-buffered saline solution (PBS). An ultrasonic bath of PBS was then performed for 3 minutes on the samples. The PBS was disposed of and another 3 minutes PBS ultrasonic bath was used. The PBS baths were used to remove the unbound BSA from the sensor.

The samples were then put on a fluorescent microscope to identify whether the BSA-FITc attached to the silicon or silicon dioxide using either TMCS or DMCS. It was determined from this experiment that the binding sequence worked better for the silicon dioxide than the bare silicon because both TMCS and DMCS bind better to silicon dioxide than silicon. This would be a problem in the final device because the exposed regions of the sensing apparatus are silicon, while the surrounding regions are silicon dioxide. If this were being used as a sensor with this chemistry, the ultimate resolution would be adversely affected because the majority of targeted analyte binding events would happen outside of the sensing region. It is imperative that the final device have a coating that is either selective to the silicon or deposited in a manner that only the nanowires are coated. However, this experiment did demonstrate that it is possible to bind to silicon so the next part of this proof of concept experiment could commence.

Following the successful results to the bovine serum albumin binding to silicon study, an additional study was performed to prove that the chemo-electrical sensing method utilizing silicon nanowires was effective. This experiment repeated the previous experiment with silicon microbars and silicon nanowires to prove the sensing technology is effective. This experiment also explored the devices' sensitivity to low concentration of the target analyte in solution.

Both nanowire and microbars were used. These samples received a CVD coating of DMCS for 15 minutes. The samples were mounted strategically so that the tape used to hold the samples during the CVD process masked the contact pads. This eliminated the possibility that the electrical property changes measured as a result of the coating and subsequent binding were from a change on the contact surface of the pads.

Electrical measurements were then taken on the samples to set up a baseline behavior prior to the exposure to the target analyte. The microbars were swept from −10 volts to 10 volts DC with the backgate held at 0 VDC, and the current that flowed through the device was measured and recorded. The microbar was also held at a 2 volt potential difference while the backgate was swept from −20 volt to 20 volts DC. The current flowing through the microbar was again measured and recorded.

After the conclusion of the electrical measurements, the sensors were placed in varying concentrations of BSA in solution. The time of exposure to BSA solution was held at a constant 20 minutes. Following the BSA soak, the samples were rinsed in PBS solution and dried with high purity nitrogen.

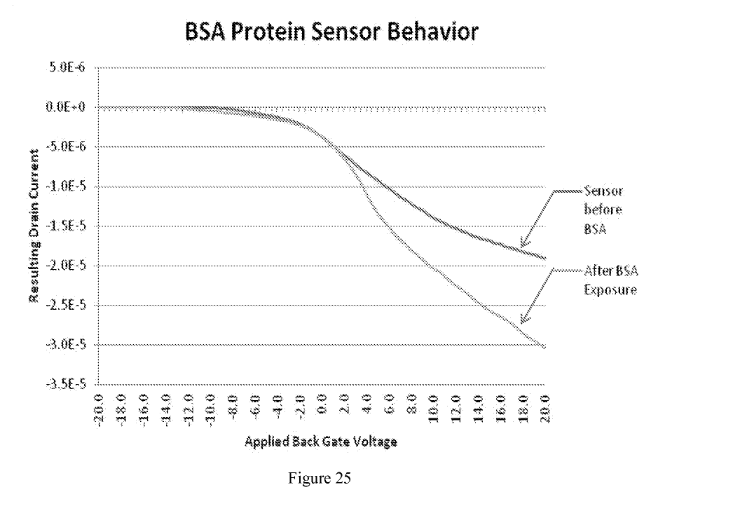

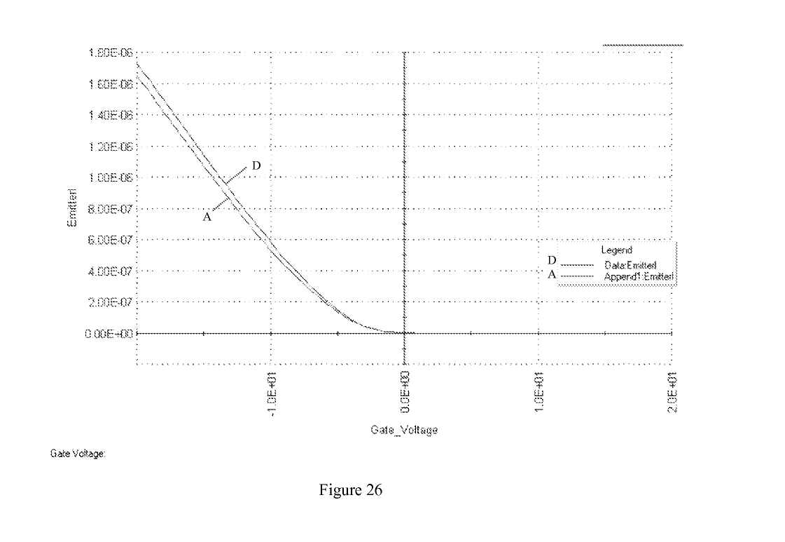

The samples were then electrically measured again using the same parameters used above. The ability to sense the BSA-FITc results of the experiments are shown in Table 9, and a graph showing the current changes with the BSA-FITc attachment is shown in FIG. 25 and FIG. 26.

TABLE 9
BSA-FITc identification sensor testing

| Concentration of | | |
|---|---|---|
| BSA-FITc in | Structure | Successful |
| solution | tested | sensing |
| 1 mg/ml | Microbars | Yes |
| 10 μg/ml | Nanowires | Yes |
| 1 μg/ml | Nanowires | Yes |
| 1 ng/ml | Nanowires | Yes |

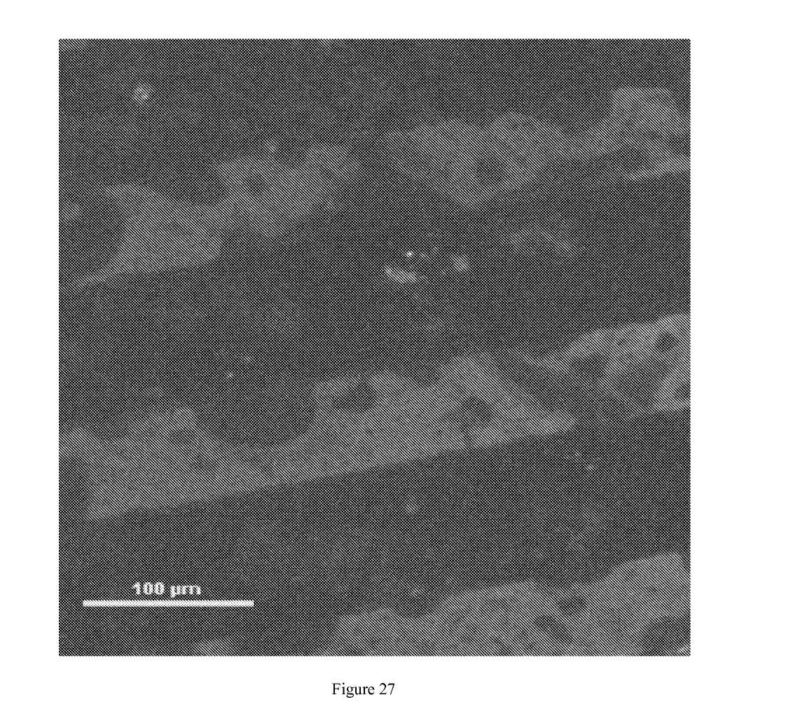

It was necessary to prove that the change in the impedance of the wires was in fact due to the existence of BSA-FITc on the wires. This was done using fluorescence to identify the location of the BSA-FITc. FIG. 27 is an image taken of one of the microbar samples with the fluorescence microscope. Note that the areas fluorescing at higher intensity are the silicon dioxide areas.

This experiment proved that it is possible to use these devices as sensing devices in a way that is similar to actual molecules of interest. The changes in current signify a great ability of the devices especially considering that the majority of the probe area cannot be sensed by the nanowires. A selective coating method is essential to bring out the maximum resolution of the sensor.

Resolution for Selective Coatings

As mentioned above, it is important for only the sensing regions of the sensor to be coated with the binding probes for the target analyte. It is possible for the probe molecule to be engineered to bind selectively to silicon over silicon oxide, but it is also possible to use deposition methods that would create the same effect. The most commonly utilized way to do this is by nanodroplet application of a probe solution onto the wire and evaporation of the solvent from the wire to leave the probe molecules bound to the nanowire. Instead of utilizing this method, in the present work electrodeposition was used for selective coating of the silicon nanowires. While the nanodroplet method is an effective means to coat the nanowires, it is a serial method that would require a significant amount of time during mass production of the sensors. Electrodeposition has the ability to selectively coat multiple wafers containing multiple nanowires simultaneously, making it far more effective a method for the goal of commercialization.

Nickel Electroplating Study

One possible solution to allow for selective binding to only the sensing areas is to put an intermediary layer between the silicon and the probe to be attached. This layer could be any number of materials, including nickel, that can be electrodeposited which would have attractive surface qualities that could be manipulated.

A study was conducted to prove that nickel is a viable option for an intermediate layer between silicon and potential probes. The deposition process chosen was electroplating because it allows for a selective coating of the nanowires, and the present study was performed to show the adhesion of electroplated nickel to silicon.

The samples for the electrodeposition study were fabricated using 4″ p-type 10 Ω/cm <100> plane silicon wafers. The wafers were RCA cleaned and 25 nm of dry thermal oxide was grown on them. They were then patterned using the inverse of the design shown in FIG. 4. The open circles were then exposed to CF4 plasma to etch them back to silicon. The substrates were then diced into individual samples, and the oxide from one of the corners was removed using hydrofluoric acid to allow for electrical contact.

The electrolyte bath used for this study is a modification of the Watts bath which has been shown to deposit finer gain sizes than standard nickel electroplating baths. The chemistry involved was nickel sulfate, boric acid and deionized water. In a large beaker, 14.062 ml of boric acid was mixed in with 454.465 ml of deionized water. After the mixture was vigorously stirred, 70.573 g of nickel chloride was added and stirred until completely dissolved in the solution.

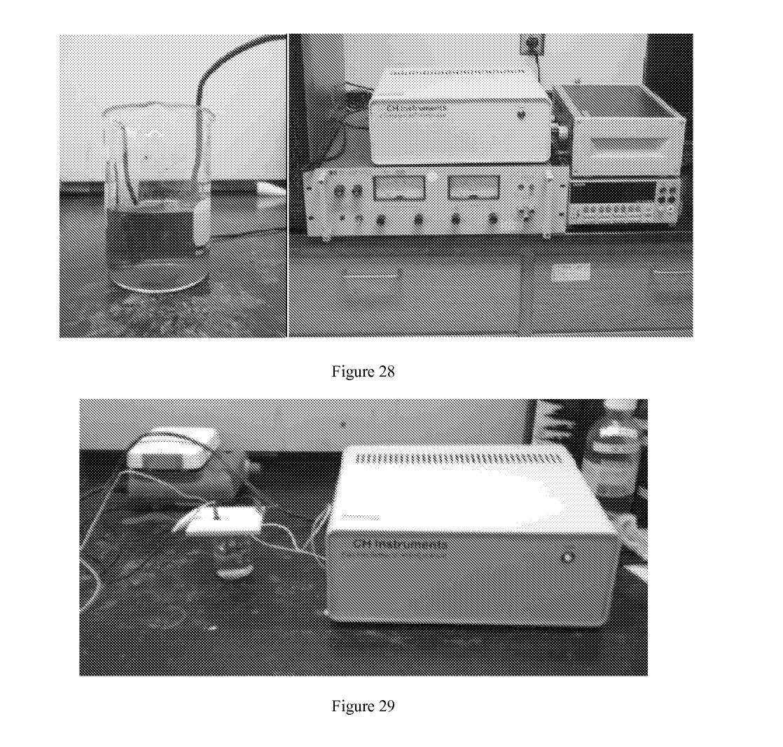

Once the solution was prepared, it was heated to 60° C. The electroplating set up for the nickel deposition can be seen in FIG. 28. The lead counter electrode was placed in the solution, and the sample was mounted to the working electrode and placed in solution. A current meter was placed in series with the electrodes, and a voltage meter was put in parallel with the voltage supply to measure both current and voltage during the deposition. The voltage source was turned on and adjusted until the current density was 0.269 mA/mm². The deposition took place for two minutes, after which the sample was rinsed in deionized water and measured on the ellipsometer. The ellipsometer confirmed that there was nickel deposition, however the thickness of the nickel was greater than the ellipsometer could measure. It was also noted that the adhesion of nickel to silicon was marginal to poor, although further adjustment of conditions is expected to improve control and adhesion issues.

Polymer Electro-Deposition

In various embodiments, binding linkers can be engineered into polymers capable of binding with molecules of interest. For example, polyaniline can be modified to contain linkers that bind or react selectively to many analytes of interest, including E. coli and glucose. Polyaniline has a conjugated polymer backbone, enabling it to transport charge through its valence hybridized bonds, but insulating to surrounding media. This material has the ability to allow for device passivation, while providing a conductive path for the change in charge after binding with the target analyte. This is the ideal scenario for the silicon nanowire chemo-electrical sensor. Not only does this material behave in a manner required for the sensing, it also can be electrodeposited; this allows for selective coating on the sensing regions and nowhere else. Ultimately, this allows for a probe material that can perform the tasks required by this material in the most effective manner.

Polyaniline can be electrodepositied because it can remain suspended in solution in its monomer form for extended periods of time. During the electrodeposition process, the monomer will cross-link into polymers at the working electrode (the substrate) through a redox process.

The research required to synthesize the selective probe polymer is both time consuming and expensive. Before effort could be justified to synthesize a probe molecule having sensitivity for E. coli, the selective adhesion of the baseline polymer to silicon using electrodeposition techniques needed to be proven. To do this, the commercially available baseline monomer, 3-aminophenylboronic acid hydrochloride salt, was used to create a monomer solution. The monomer solution used for this experiment is a modification of the procedures of Shoji (E. Shoji, M. S. Freund, “Potentiometric Saccharide Detection Based on the pKa Changes of Poly(aniline boronic acid),” J. Am. Chem. Soc., Vol. 124, pp. 12486-12493, 2002):

The chemicals used in creating the monomer solution were 3-aminophenyboronic acid hydrochloric salt, hydrochloric acid, sodium fluoride, and Nafion solution. The chemistry was mixed in a 50 ml beaker. Next, 12.5 ml of 0.2M hydrochloric acid was added to the solution and stirred at 100 RPM at room temperature. While stirring continued, 87 mg of 3-aminophenyboronic acid hydrochloric salt was added to the solution. After the salt was completely dissolved, 21 mg of sodium fluoride was added to the solution, followed by 2 ml of Nafion solution. After all chemicals were completely dissolved into the solution, the monomer solution was stirred for 30 min.

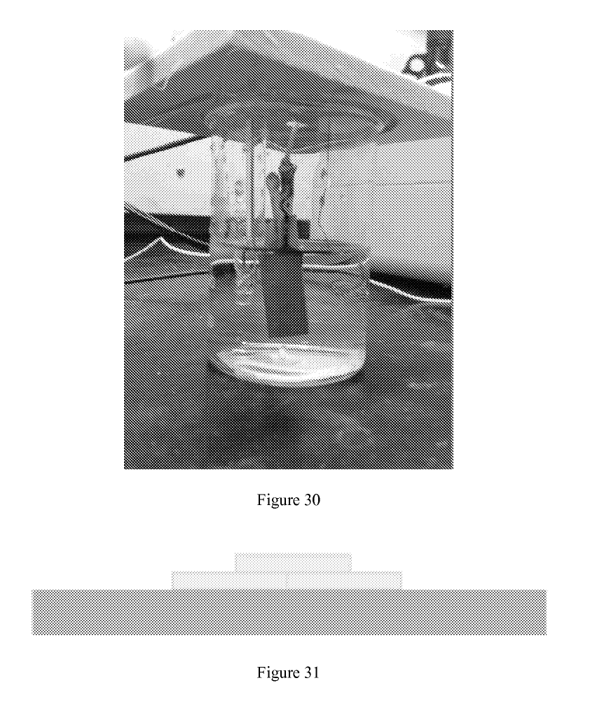

The electrodeposition work was conducted using a CH Instruments 660 E potentiostat. The setup used on all of the following experiments was a three electrode setup (FIGS. 29, 30). This allowed for excellent control. The electrodes consisted of a working electrode, the substrate connected by clip, the counter electrode, a platinum coil, and a commercial reference electrode, CH Instruments model CHI111, made of silver coated with silver chloride. The counter electrode and reference electrode were chosen to eliminate reactivity with the monomer solution.

The driving source for the electrodepostion was a cyclical voltage signal. The voltage was applied in a saw tooth waveform between 0 and 1.1 volts at 1 Hz. Due to the potential barrier set up by the steel clip and the silicon, the actual voltages seen by the solution were approximately 0 to 0.9 volts. The current during the deposition was measured to establish how the current density affects the deposition rate. This is a value which has not been extensively studied by other groups. The deposition was concluded when the charge on the working electrode reached 10 μC.

The samples for the proof of concept for the study of electrodeposition onto silicon were fabricated using 4″ p-type 10 Ω/cm <100> plane silicon wafers. The wafers were RCA cleaned and then cleaved into 1 cm by 4 cm pieces. Prior to electrodeposition, the samples were placed in 50:1 deionized water to hydrofluoric acid to remove the native oxide layer.

After the native oxide was removed the samples were placed into the monomer solution and the electrodes were inserted as shown in FIG. 30. The cyclic voltage was applied through the potentiostat until the charge on the working electrode reached 7.5 PC. The sample was then removed from the monomer solution and rinsed in a deionized water bath. Following the rinse, the sample was put into a two electrode bath of 0.1 M hydrochloric acid. A DC voltage was held at 0.8 volts for 10 seconds. This step assures that the binding process is completed. The sample was then put into a deionized water bath and rinsed. Following this step, the sample was placed into a bath of 7.4 pH PBS for 24 hours. This ensures that the reaction is complete, and helped to remove the uncross-linked material.

Sample measurements were conducted after the deposition of the polymer using the ellipsometer. The modeling method utilized for describing the optical properties of the polymer was the Cauchy approximation method. This method assumes a functional relationship between the index of refraction (n) and the extinction coefficient (k) that can be represented by a minutely changing function of wavelength (λ). The model included with our ellipsometer software package also allowed for the inclusion of an absorption tail. Equations 1 and 2 show the relationship between the index of refraction and wavelength, and the relationship between the extension coefficient and wavelength, respectively.

$$\begin{matrix}n ( λ ) = A + \frac{B}{{λ}^{2}} + \frac{C}{{λ}^{4}} & ( 1 ) \\ k ( λ ) = α * {e}^{12400 β ( \frac{1}{λ} - \frac{1}{γ} )} & ( 2 )\end{matrix}$$

In the above equations, α is the coefficient amplitude, β is the exponent factor, γ is the band edge, and A, B, and C are constants. During the model fitting process, A, B, C, α, and β were allowed to be manipulated by the software to fit the model output to the experimentally measured data. This allowed the software to minimize the MSE value for the model fit discussed above.

After the experimentation was completed, it was determined that the polymer was successfully deposited onto the silicon. The film was modeled as a 5.6 nm thick polymer layer. This is no more than three atomic layers of this polymer. To test the adhesion, the “scotch tape” method was utilized; a piece of scotch tape was adhered to the polymer film and then ripped off. The sample was then measured again using the ellipsometer. No change in thickness was observed.

Selectivity of Polyaniline Electrodeposition

Following the successful completion of the proof of concept of polyaniline binding to pure silicon, the selectivity of binding of the polymer by electrodeposition was tested.

As explained above, the silicon nanowires are surrounded by silicon oxide. Only the silicon is sensitive to signal changes caused by binding events. Therefore, if the analyte were to bind to a probe that was attached to an insensitive region this would be detrimental to the ultimate resolution of the system.

The sample choice for this experiment was glass slides. The glass slides were cleaned using acetone and isopropyl alcohol. Glass slides were then loaded into a sputtering chamber, arranged in a shadow masking pattern to allow for half of the two bottom slides to be coated. A diagram of the cross section of the slides to show the configuration is shown in FIG. 31. Silicon was then sputter deposited to a thickness of 95 nm.

Following deposition, the samples were measured using ellipsometric techniques to get a baseline thickness of the silicon and the glass slides prior to the electrodeposition. Due to the substrates being transparent in nature, a piece of scotch tape was adhered to the back to help cancel out backside reflections during the measurement. In order to ensure that the selectivity values were accurate, the two bottom slides had both the silicon and the undeposited area of bare glass measured.

The samples were then individually dipped in 50 to 1 deionized water to hydrofluoric acid for 20 seconds to remove the native oxide. The slides were then inserted into the Polyaniline monomer solution described in the experiment above and the electrodeposition took place using the parameters described above. The only deviation from the process in the last experiment was the cutoff charge for this experiment was 10 μC. The exposed silicon area was approximately the same as the previous experimental samples; the deposition was allowed to run for a longer period of time to increase the deposition thickness to better identify the polymer film with the ellipsometer. The samples were rinsed following the completion of the electrodeposition, and then exposed to the same hydrochloric treatment to finish the redox reaction. Following another deinonized water rinse, the samples were placed in PBS solution for 24 hours.

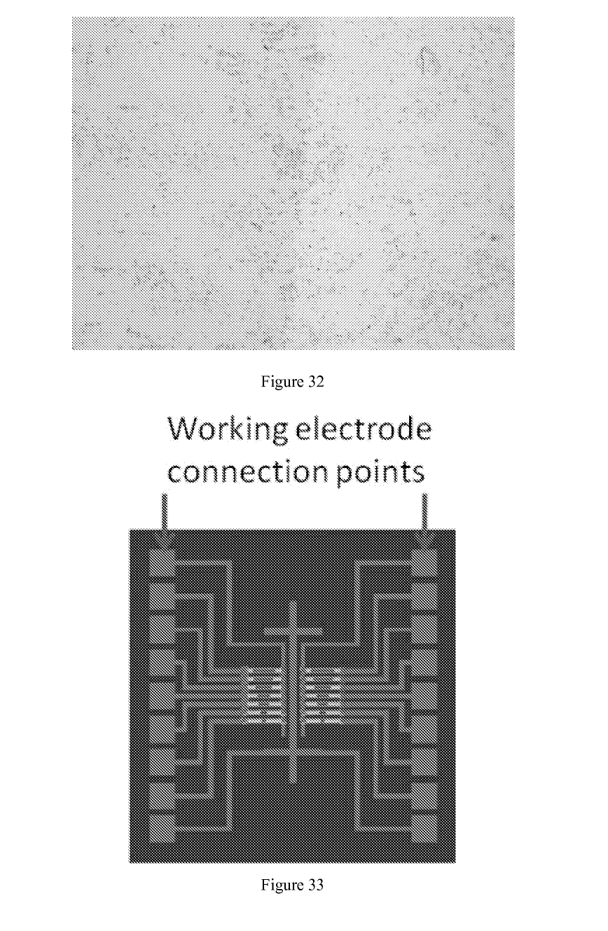

Ellipsometric measurements were then taken to identify changes in the device film stack. The results are shown in Table 10. As shown in Table 9, this experiment confirmed that the polymer only deposits on the silicon (see also FIG. 32). No deposition took place on the Silicon oxide.

TABLE 10
Electrodeposition selectivity experiment results

| | | Pre-Dep film | | Post-Dep film | |
|---|---|---|---|---|---|
| | Material | thickness | | thickness | |
| Bare Glass | SiO₂ | 1 | mm | 1 | mm |
| | Polymer | 0 | nm | 0 | nm |
| Silicon | SiO₂ | 1 | mm | 1 | mm |
| | Si | 95 | nm | 95 | nm |
| | Polymer | 0 | nm | 8.6 | nm |

Polyaniline Coating Silicon Microbars

Following the results of the previous experiment showing that the electrodeposition process for polyaniline has the ability to selectively coat silicon, an additional experiment was conducted to show that coating to smaller features is also possible.

For this experiment, electrical measurements were used to measure changes in the conduction to prove binding. However, the feature size of the microbars was too small to use ellipsometry to prove the existence of the polymer after the deposition process was completed.

The samples for this experiment were silicon microbars with metal electrical contacts. The ground lead of the sample had a wire soldered to it to allow for all of the nanowire to be coated simultaneously with polyaniline. The contact points are shown in FIG. 33.

Prior to deposition of the polymer, the samples were measured using a −10 to +10 volt DC sweep with a 0 volt backgate bias. They were also measured with a 5 volt potential across the microbar structure and a −20 to +20 volt DC sweep across the backgate. For both measurements, the current across the drain was measured and recorded.

Following the electrical measurements, the sample was inserted into a 3-probe bath of the polyaniline monomer solution. The voltage was cyclically swept from 0 volts to 1.1 volts; there was a potential barrier drop in the voltage set up by differences in work functions of the silicon and the contact metal so the voltage seen in the solution was actually 0 volts to 0.7 volts. The voltage was applied in a cyclical sawtooth waveform at 1 Hz until the charge on the sample reached 10 μC.

The sample was removed from the monomer solution and immediately rinsed in deionized water. Following the rinse, a 10 second 0.8 volt potential was applied to the substrate in a 0.1 M hydrochloric bath to finish the redox reaction. The sample was removed following the completion of this step and rinsed in deionized water. A room temperature bath of PBS for 24 hours was used to finish the sample coating and remove the existing monomer chains.

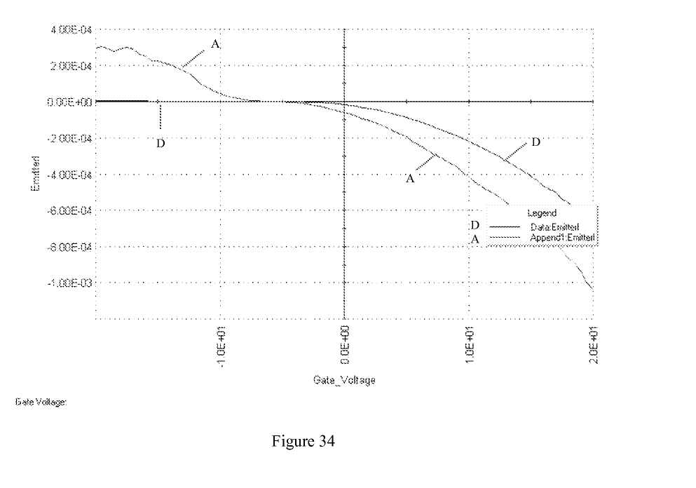

After the completion of the coating process, the samples were then electrically measured using the same parameters as described earlier. The results of before and after the deposition are shown in FIG. 34. It was assumed that the change in behavior is caused by the application of the probe film, which is reasonable in view of the results of the previous polyaniline experiments. The blue line is the measurement of the drain current before the polymer deposition; the purple line is after the deposition.

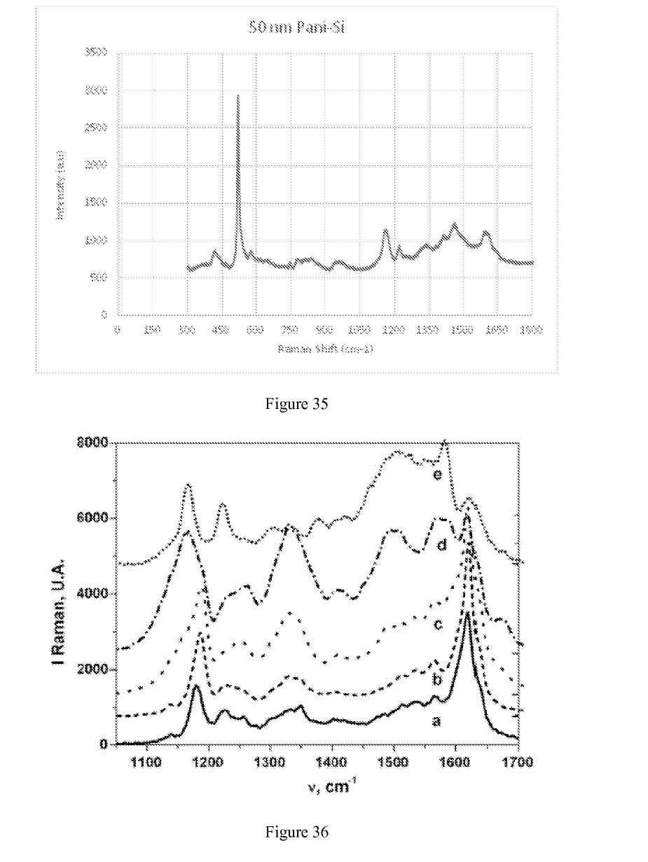

FIGS. 35 and 36 show Raman spectroscopic data to confirm the deposition of polyaniline on the silicon. Note that the units are arbitrary units (A.U.'s) so the absolute values are not important, but the shapes of the graphs are. FIG. 35 shows test data taken using Raman spectroscopy techniques of a 50 nm polyaniline layer electrodeposited onto <100> silicon. FIG. 36 shows Raman data for polyaniline films.

Nickel plating was utilized to show that electrodeposition methods are feasible. Following this, a polymer deposition was performed and test both ellipsometrically and electrically. Either of these methods provide an easy means of coating several wafers of devices at one time when scaling up the disclosed processes.

### Example 3

### Characterization of Silicon Nitride Films

The following example describes the preparation of silicon nitride thin films by low-pressure chemical vapor deposition (LPCVD) and by radio frequency (RF) sputter deposition, which may be utilized in the top down fabrication of sub-70 nm silicon nanowires for biochemical sensing with functionalization. A series of experiments were performed to characterize the suitability of the films in the overall fabrication of the nanowires. It was observed that the sputtered silicon nitride had to be thicker than the LPCVD silicon nitride to serve as a sufficient masking layer. However, the higher density LPCVD film required a longer etch duration. The silicon nitride thin films were analyzed through a series of chemical etching, oxidation, and ellipsometric measurements. It was found that the sputtered nitride film serves as an effective barrier film for top down nanowire fabrication.

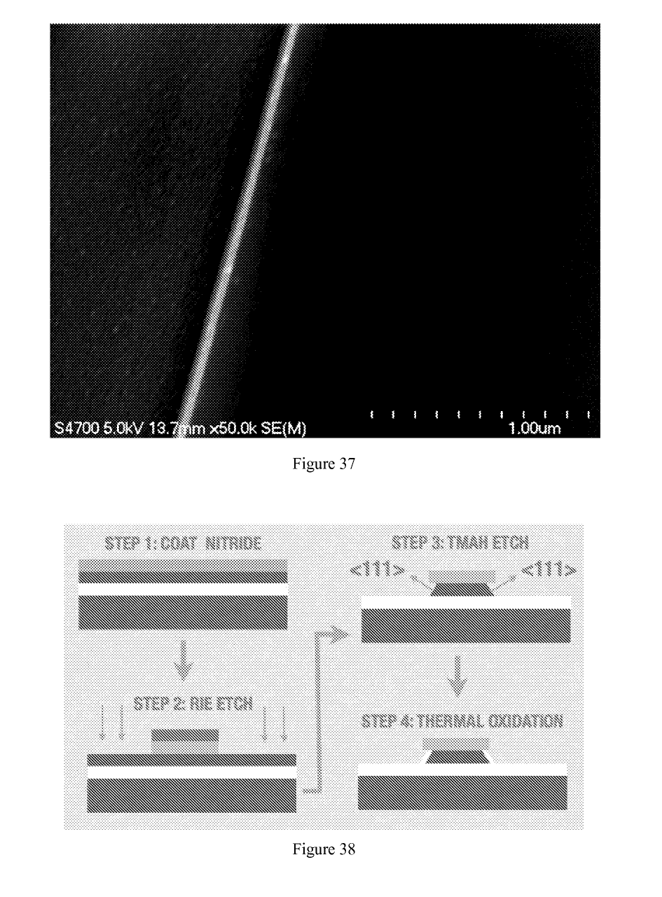

The top down approach for silicon nanowire nano-fabrication is useful in biosensing applications due to its high yield, low cost and consistency in device production. The dimensions of a silicon nanowire, as shown in FIG. 37, allow femtomolar level sensitivity of biological species detection, which then enables early detection of diseases such as cancer. There are many different process flows that are commonly used to fabricate sub-70 nm silicon nanowires, including a method using lithography to pattern a 2 μm line hard mask, then anisotropic etching, repetitive oxidation and buffered hydrofluoric acid wet etching to reduce the dimensions of the silicon nanowire; and a method using conventional micro-fabrication technologies including micro-lithography, oxidation, and wet anisotropic etching. These two methods are the references for this work because wafer scale batch fabrication remains a more feasible route for the commercialization of biosensors.

FIG. 37 is a scanning electron microscopic (SEM) image of an exemplary single strand of silicon nanowire fabricated according to methods of Du et al (H. Du, R. E. Tressler, K. E. Spear, and C. G. Pantano. “Oxidation Studies of Crystalline CVD Silicon Nitride.” J. Electrochem. Soc., vol. 136, no. 5, pp. 1527-1536, May 1989). The width of the nanowire is approximately 70 nm, with its length ranging from 1 μm to 1000 μm.

Silicon nitride (Si₃N₄) film can be utilized as an efficient diffusion mask in device passivation and selective doping, and in the selective oxidation of silicon for CMOS and MEMS, due to the slow nature of oxygen diffusion through the film and the slow oxidation of Si₃N₄ itself. Herein, Si₃N₄ film is used as a diffusion and etch mask for a device silicon layer of a silicon on insulator (SOI) substrate during the thermal oxidation process. Ultimately, this process path produces silicon nanowire devices for biological sensing.

FIG. 38 illustrates the first 4 of 10 process steps used in conventional silicon nanowire fabrication, in a cross sectional view. The detailed process flow is known to persons of skill in the art; only issues related to the first four steps and the removal of the Si₃N₄ are discussed herein. The first process step was deposition of the Si₃N₄ film on the silicon on an insulator (SOI) substrate. This was followed by a lithography step to pattern the Si₃N₄ layer. Reactive ion etching (RIE) in Step 2 exposed the silicon device layer so that the exposed device silicon could be removed in a 25% TMAH solution at 50° C. in Step 3. The anisotropic etchant removes silicon along the (100) plane approximately 100 times faster than along the (111) plane, allowing an etch stop along the (111) planes. A 950° C. dry thermal oxidation at 1 atm. was performed in Step 4 to form the silicon oxide (SiO₂) protective sidewalls on all exposed (111) planes of silicon prior to the removal of the Si₃N₄ film.

There are different methods known for depositing Si₃N₄ thin films, including sputtering, low-pressure chemical vapor deposition (LPCVD), reactive evaporation, pulsed laser ablation, and plasma enhanced chemical vapor deposition (PECVD). For the silicon nanowire process discussed herein, the Si₃N₄ film exhibited three properties which aided the fabrication process. First, the Si₃N₄ was an effective diffusion barrier during the dry oxidation step; second, an etch mask was used during a tetramethylammonium hydroxide (TMAH) chemical etch; and finally, the film was easily dissolvable in phosphoric acid (H₃PO₄). These properties aided the process so that the resulting silicon nanowire dimensions remained well controlled and uniform.

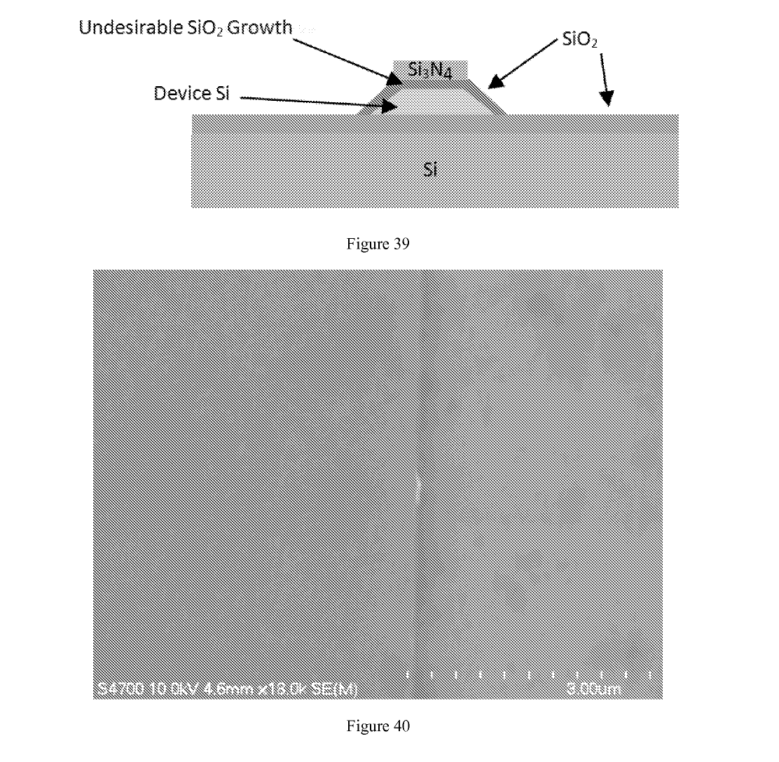

The diffusion barrier behavior is of interest in nanowire formation because an insufficient amount of nitride film may allow for the oxidation of the top surface of the device silicon, as illustrated in FIG. 39. In order to anisotropically etch the device silicon with TMAH in the subsequent step, a longer HF dip would be needed to remove the oxidized silicon layer, potentially compromising the integrity of the sidewall oxide. This may allow the device silicon to become completely etched away because there is no etch stop on the exterior (111) plane.

A FE-SEM image of the silicon nanowire etched away from an insufficient SiO₂ film is shown in FIG. 40. There is a small area where the device silicon is left in this image, where the SiO₂ had adequate ability to stop the TMAH etch. In order to make the TMAH etch a repeatable process, a determination of which method of deposition would be useful to meet the processing requirements was made, and the results are discussed herein.

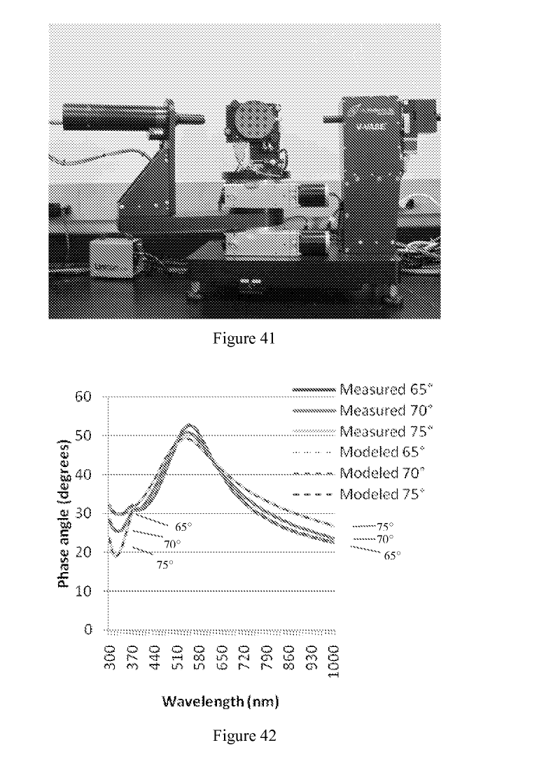

The two methods investigated in this example were RF sputter deposition and LPCVD. The Si₃N₄ films that were explored were sputter deposited by a 99.99% pure Si₃N₄ target using a Perkin-Elmer Randex Sputtering System Model 2400, and LPCVD deposited using dichlorosilane (SiCl₂H₂) and ammonia (NH₃) at 800° C. To aid in the comparison of the characteristics of the two films, the three desired qualities of the Si₃N₄ films were tested independently, thus different experiments were performed with each film type. After determining the characteristics of the films, an additional experiment was performed to find the stoichiometry of the Si₃N₄ film that was useful for the requirements of this project. The film stoichiometry was measured using a J. A. Woollam VVASE 400 ellipsometer, shown in FIG. 41.

The ellipsometer measures the change in both polarization and light intensity during a sample scan. That data may then be compared to a model using known film types. For the experiments described herein, an effective medium approximation layer (EMA) was used to integrate the different silicon nitride layers into the model. This layer was used to calculate the optical properties of a compound material. For the experiments performed herein, the EMA layer allowed for different compositions of the silicon nitride films to be accurately represented in the models and also allowed for the exact stoichiometry to be measured in the films.

The ellipsometry measurements were conducted from 300 nm to 1000 nm in 10 nm steps from 65 degrees to 75 degrees incident angle in step sizes of 5 degrees. This data was then modeled using a 500 m silicon layer, a SiO₂ layer, and an EMA layer mixing Si₃N₄ and decoupled silicon. The percentage of silicon and stoichiometric silicon nitride in the EMA layer, the film thickness of the SiO₂ layer and the EMA layer were set as variables in the model. This allowed software to manipulate these values to fit the model to the scan data. One of the resulting model fits to scanned data is shown in FIG. 42.

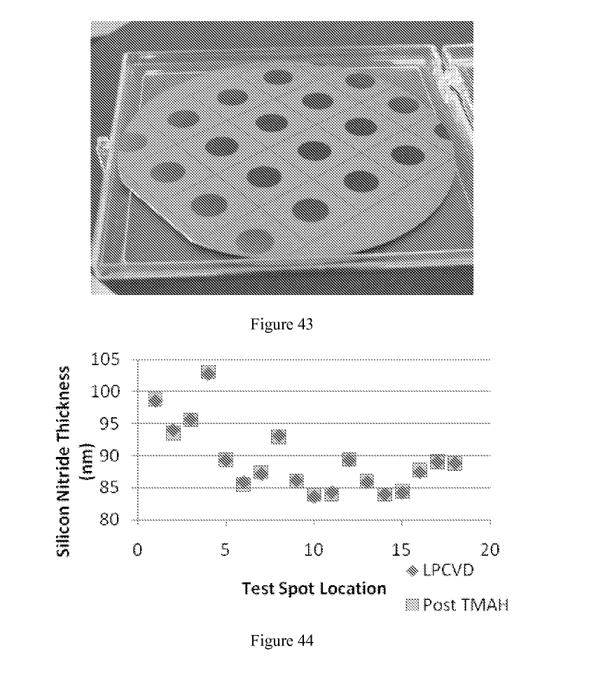

The first experiment evaluated the ability of each deposition method to limit the diffusion of oxygen. The goal was to determine a useful thickness for each type of Si₃N₄ film. Samples were prepared by depositing Si₃N₄ on 4 inch (100) p-type silicon wafers. The sputtered Si₃N₄ was deposited to a thickness of 180 nm and the LPCVD Si₃N₄ was 102 nm thick. The samples were then lithographically patterned and RIE etched to create sixteen 1 cm diameter circles that enabled the ellipsometry measurements, as shown in FIG. 43. The circles were then selectively etched to allow for a 3 nm step size from 6 nm to 102 nm for the LPCVD samples and a 5 nm step size from 100 nm to 180 nm for the sputtered films. All samples were then measured with the ellipsometer to confirm the Si₃N₄ thicknesses. The samples were then placed in a dry oxidation ambient (O₂) for 15 minutes at 950° C. The samples were then measured again on the ellipsometer to determine if a silicon oxide layer had grown beneath the Si₃N₄ film.

The second experiment tested the etch rates of both film types in TMAH at 50° C. and H₃PO₄ at 150° C. Samples were prepared by depositing 100 nm of Si₃N₄ on 4 inch (100) plane p-type silicon wafers. During the sputtering process, nitrogen was introduced into the argon gas to give the sputtered Si₃N₄ film the same stoichiometry as the LPCVD film. To confirm this, the samples were measured on the ellipsometer after deposition was completed.

The samples were then lithographically patterned and RIE etched to create sixteen 1 cm diameter circles. The photoresist was then removed, and measurements were taken with the ellipsometer. Then a six second 10:1 deionized water to 49% hydrofluoric acid dip was performed. The sample was rinsed and then placed in TMAH at 50° C. for 10 minutes. The sample was rinsed and then measured with the ellipsometer to determine the film's masking effectiveness.

The same sample development process was used for the H₃PO₄ etch rate testing. After the pre-etching measurements were taken, the LPCVD samples were dipped in H₃PO₄ at 150° C. for 5 minutes and the sputtered samples were dipped in H₃PO₄ at 150° C. for 2 minutes. The etch rates were then found by measuring the samples with the ellipsometer.

An experiment was also carried out to determine the impact of the stoichiometry of the RF sputter-deposited nitride films to better correlate fabrication properties with the LPCVD films. By changing the ratio of nitrogen to argon gas, the series of samples described in Table 11 were fabricated. After deposition, the samples were lithographically pattered and RIE etched to create sixteen 1 cm diameter circles. The photoresist was then removed, and measurements were taken with the ellipsometer. A six second 10:1 deionized water to 49% hydrofluoric acid dip was performed. The sample was rinsed and then placed in TMAH at 50° C. for 10 minutes. The sample was rinsed and then measured with the ellipsometer to determine the film's masking effectiveness. If there was no etching of the Si₃N₄ film, the samples were then dipped in H₃PO₄ at 150° C. for 2 minutes. The etch rates were then found by measuring the samples with the ellipsometer.

TABLE 11
SPUTTERED SAMPLES FABRICATED WITH
DIFFERENT GAS RATIOS

| | N2:Ar Ratio | Stoichiometry |
|---|---|---|
| Sample 1 | 0:1 | 29.7% Si rich |
| Sample 2 | 3:200 | 1.9% Si rich |
| Sample 3 | 1:19 | 2% N rich |
| Sample 4 | 3:17 | 7.9% N rich |
| Sample 5 | 4:16 | 9% N rich |

The combination of the oxidation diffusion barrier thickness of each film type with the etch rates in both TMAH and H₃PO₄ were considered to determine the optimal processing technology for this sensor device fabrication. The minimal thicknesses for an effective oxygen diffusion barrier were found to be about 96 nm for the LPCVD film and about 138 nm for the sputtered silicon nitride film.

Next, we confirmed that both films were an effective masking layer in TMAH by verifying that there was no etching of the nitride films in the TMAH anisotropic etchant. It was found that all samples had excellent resilience to the TMAH solution. FIG. 44 shows the ellipsometric model data of one of the LPCVD samples before and after the etching process. The error on the thickness measurements from the ellipsometer were plus or minus 1 nm. The graph shows the measurements are within that error tolerance, thus it can be reasonably determined that the silicon nitride was not etched.

Finally, the samples were exposed to H₃PO₄ at 150° C. The etch rates for all steps are shown in Table 12.

TABLE 12
LPCVD AND SPUTTERED SILICON NITRIDE

| | | Diffusion | |
|---|---|---|---|
| | TMAH | Mask | |
| | Etch Rate | Thickness | H3PO4 Etch Rate |
| CDV | 0 nm/sec | 96 nm | 1.2 nm/min |
| Si₃N₄ | | | |
| Sputtered | 0 nm/sec | 138 nm | 6.6 nm/min |
| Si₃N₄ | | | |

The CVD film is a good diffusion barrier during the dry oxidation, and is an effective hard mask during the TMAH dip. However, it is more difficult to remove than the sputtered film. It would take approximately 80 minutes to fully etch the 96 nm Si₃N₄ required to make a good diffusion barrier. H₃PO₄ also etches SiO₂, but at a rate approximately an order of magnitude slower (see, e.g., W. van Geldger and V. E. Hauser. “The Etching of Silicon Nitride in Phosphoric Acid with Silicon Dioxide as a Mask”. J. Electrochem. Soc. 114, no. 8, pp. 869-872, August 1967, pp. 869-872). However, there is only 25 nm of SiO₂ on the (111) plane of the silicon nanowires. With an etch as long as what was needed for the LPCVD nitride, it may endanger the silicon nanowire sidewall's SiO₂ integrity to remain an effective etching mask to TMAH. It is possible to speed up the etch using a higher temperature, but this may cause the concentration of the H₃PO₄ to be higher than 85% by boiling some of the water off. The higher concentration decreases the etch selectivity of silicon nitride to both silicon oxide and silicon (van Geldger et al.); both of which may be undesired in the chosen process flow. For this reason, it was determined that the LPCVD silicon nitride may not be the best choice for this process flow.

The sputtered nitride was found to be a good diffusion barrier for thicknesses above about 138 nm, is an effective etch mask during the TMAH etching, and is easier to remove (at 6.6 nm/min) than the LPCVD film. For a full sample, it would take approximately 21 minutes to fully remove the nitride. Though the sputtered silicon nitride was thicker than the LPCVD silicon nitride, the lower density allowed for quicker removal during the H₃PO₄ etch. This quicker etching may reduce the chance of damaging or removing the silicon nanowire sidewall SiO₂ and may also allow for a thinner silicon mask when selectively etching the silicon nitride. For these reasons, sputtered silicon nitride was chosen as a more preferred film for this process flow.

After analyzing fabrication methods for the silicon nanowire process path, an additional study was conducted to find a stoichiometry of the silicon nitride films that would decrease the etch time in H₃PO₄ while retaining the other beneficial qualities of this process sequence. Varying the N₂:Ar ratio during the sputter deposition resulted in variations in the stoichiometry and resulting etch characteristics, as shown in Table 13.

TABLE 13
SPUTTERED SAMPLES FABRICATED WITH DIFFERENT GAS
COMBINATIONS AND THEIR ETCH RATES

| | N₂:Ar | | TMAH | H₃PO₄ Etch |
|---|---|---|---|---|
| | Ratio | Stoichiometry | Etch Rate | Rate |
| Sample 1 | 0:1 | 29.7% Si rich | 0 nm/sec | 5.5 nm/min |
| Sample 2 | 3:200 | 1.9% Si rich | 0 nm/sec | 9 nm/min |
| Sample 3 | 1:19 | 2% N rich | 0 nm/sec | 11.9 nm/min |
| Sample 4 | 3:17 | 7.9% N rich | 0 nm/sec | 19 nm/min |
| Sample 5 | 4:15 | 9% N rich | 0 nm/sec | 21.7 nm/min |

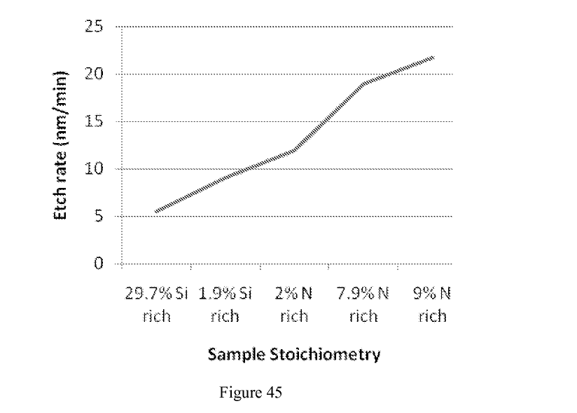

The first two rows in Table 13 show the chemical makeup of the sputtered films with the different gas flows used during fabrication. When argon was used alone, it was observed that the Si₃N₄ film was 29.7% silicon rich. This sample had the lowest etch rate in the H₃PO₄. As the nitrogen was introduced to the argon gas during deposition, the etch rate in H₃PO₄ increased. The etch rates for the different stoichiometries are shown in FIG. 45.

As shown in FIG. 45, the 9% nitrogen rich sample had the fastest etch rate. This correlates with the 1:4 nitrogen to argon gas ratio during the sputtering process. Due to its high etch rate in H₃PO₄, the ability to maintain an effective etching mask in TMAH, and its properties as a diffusion barrier during dry oxidation, this is an effective deposition method for the silicon nitride films examined herein.

The goal of this experiment was to understand and characterize the silicon nitride films for a top down silicon nanowire fabrication process. The oxidation analysis investigated the thickness of the silicon nitride films to maintain an effective diffusion barrier. The results showed that LPCVD silicon nitride had a minimum thickness as a diffusion barrier at about 96 nm and the RF sputtered silicon nitride at about 138 nm. The etch rate study found that both LPCVD and RF sputtered films had the ability to mask TMAH, and that the RF sputtered film etches at a much higher rate than the LPCVD film in hot H₃PO₄. Combining the data from these experiments, it was concluded that the RF sputtered silicon nitride film was an effective choice for the silicon nanowire process flow due to the shorter duration required in H₃PO₄ to fully remove the film.

An additional analysis was conducted to evaluate the stoichiometry of the silicon nitride films. It was concluded that a 9% nitrogen rich sample works well for the silicon nanowire fabrication process. This experiment has contributed to the understanding of silicon nitride characteristics for nanoscale feature fabrication, and will improve the quality and uniformity of the production of top down silicon nanowires.

### Example 4

### Silicon Nanowire Device Behavior

A single silicon nanowire sensor (e.g. one of the sensors of an array) behaves akin to a MOSFET device. In this device, the nanowire functions as a channel in which current can flow from one end of the nanowire to the other. The amount of current flowing depends on the voltage potential between the two ends of the wire and the number of free carriers in the wire. The number of free carriers is affected by the nature of the material itself, the charge of any molecule bound to the nanowire, and the capacitive effects of the backgate potential (any voltage applied to the backside of the sensor will change the conduction of the wire). Using the silicon nanowire as a sensor depends on the ability to detect changes in the free carriers while keeping the voltage potential on the nanowire and the backgate the same.

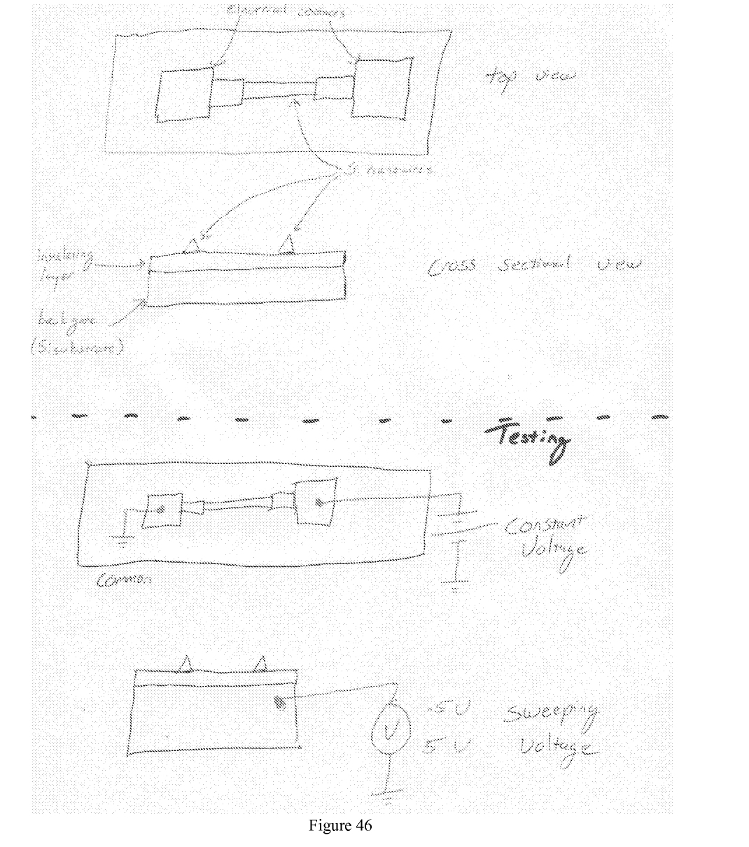

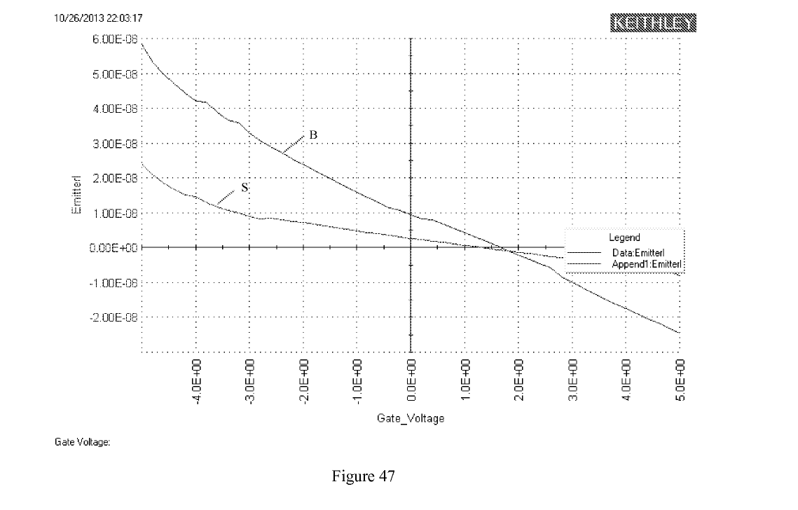

Testing to date has been conducted using a Keithley 4200 semiconductor characterization system. This unit utilizes source measurement units (SMU's) which have the ability to drive currents and voltages, as well as measure currents and voltages simultaneously. The parameters of the driven voltage or current can be controlled via a Windows interface, and the measurements are taken and graphed (see, e.g. FIG. 47). Typically for the nanowire and microbar sensors, a voltage sweep is performed on the backgate and the nanowire is held at a slant voltage potential. FIG. 46 shows a diagram of a nanowire sensing system (top) and wiring for electrical measurements from the system (bottom).

A solution of analyte is mixed up from dry powders. The concentration and analyte in solution is set depending on the testing being performed. Using the Keithley 4200, a baseline voltage/current measurement is taken as described below. The sensor is then exposed to the solution for 10-15 minutes depending on the test being performed. Following the “incubation” period, the sensor is rinsed in phosphate buffered solution (PBS) and dried using compressed 99.99999% nitrogen. The sample is then measured again using the Keithley 4200 to find the difference in voltage/current characteristics. The rinsing of the sample in PBS in not necessary for testing, but it does allow for confirmation that only bound analytes are tested for (important for the selectivity testing that is underway) and the presence of the proper analyte can verified using fluorescent imaging.

Prior to a sample solution (which may contain the target analyte of interest) being applied to the sensor, a constant voltage potential is held on one end of the nanowire, and the other end is held at ground (creating a slant voltage potential) and a voltage sweep is conducted on the backgate of the sensor. This sweep changes the number of free carriers available to transport current through the silicon nanowire due to capacitive effects on the wire. This initial test provides a baseline of the attributes of the wire; subsequent measurements of the nanowire that are made after the solution containing target analyte is applied are compared to this baseline.

Upon contact of the aqueous solution with the coated nanowire, target analyte in the solution binds to probe molecules attached to the nanowires. When a binding event takes place between a probe molecule and a target analyte, the number of free carriers available for charge transfer in the wire changes, resulting in a measurable change in current when the voltage difference across the nanowire is held at a constant. The parameters used on the initial baseline electronic testing used above are repeated after application of the test sample to the nanowire, and the difference between voltage and current are measured; changes indicate that there is target analyte present in the solution. Secondary means of verification, including positive and negative controls on the sensor, fluorescence tagging of analytes, and spectroscopic measurements have been utilized to verify electrical results in the testing conducted to date.

FIG. 47 shows nanowire sensing of E. coli. The blue line (B) was taken before exposure and the purple (S) line was taken after exposure to picomolar levels of E. coli. Measurements were taken on a nanowire sensor coated with goat anti E. coli O157:H7. The measurements were taken using a constant voltage slant of 5 volts held across the nanowire, and the voltage of the gate was swept for −5 to +5 volts in respect to the common on the nanowire.

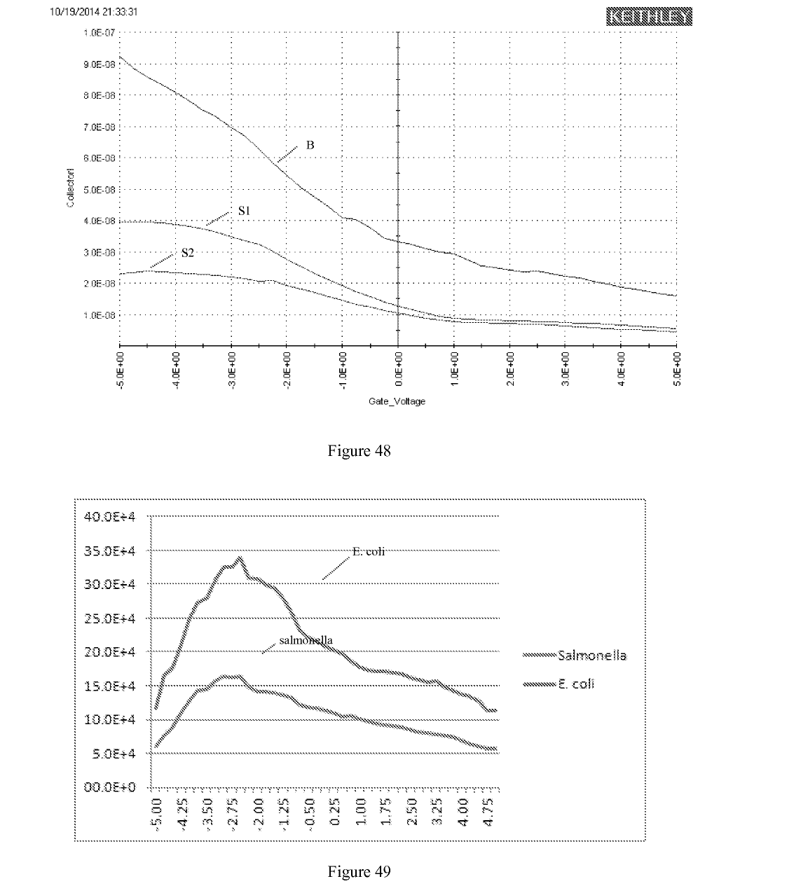

FIG. 48 shows nanowire sensing of salmonella. The blue line (B) was taken using a nanowire sensor coated with goat anti salmonella before exposure to sample, and the purple lines were taken from the same nanowire after exposure to picomolar levels of salmonella. The measurement was taken using a constant voltage slant of 10 volts held across the nanowire, and the voltage of the gate was swept for −5 to +5 volts in respect to the common on the nanowire. The two purple lines (S1, S2) depict two different concentrations of analyte to which the nanowire was exposed.

FIG. 49 shows selectivity data for salmonella and E. coli using negative and positive controls. The two traces on the graph in FIG. 49 show the ratio of signal change between 1 mg/ml concentrations of positive and negative controls. The high concentration was chosen to ensure full saturation of the sensor by the target analyte, and allow the same to happen with non-target if non-specific detection occurred. The signal change caused by respective negative controls was well within the background noise of the sensor. Negative controls used for the testing were E. coli O157:H7 for the Salmonella sensor and E. coli 045 for the E. coli O157:H7 sensor.

Various features and advantages of the invention are set forth in the following claims.

1. A method for fabricating silicon nanowires, comprising the steps of:
depositing a silicon nitride layer on a silicon on insulator (SOI) starting wafer;
patterning the silicon nitride to define at least one silicon microbar;
etching the SOI starting wafer to expose the at least one silicon microbar, wherein the at least one microbar is surrounded by a raised perimeter;
growing a silicon oxide layer on the raised perimeter of the at least one microbar; and
etching a portion of the at least one silicon microbar to produce at least one silicon nanowire adjacent the silicon oxide layer.

2. The method of claim 1, wherein depositing a silicon nitride layer on a silicon on insulator (SOI) starting wafer comprises depositing a silicon nitride layer on a silicon on insulator (SOI) starting wafer by sputtering.

3. The method of claim 1, wherein patterning the silicon nitride to define at least one silicon microbar comprises patterning the silicon nitride to define at least one silicon microbar using UV photolithography.

4. The method of claim 1, wherein etching the SOI starting wafer to expose the at least one silicon microbar comprises etching the SOI starting wafer to expose the at least one silicon microbar using TMAH.

5. The method of claim 1, wherein growing a silicon oxide layer on the raised perimeter of the at least one microbar comprises growing a silicon oxide layer on the raised perimeter of the at least one microbar using thermal oxidation.

6. The method of claim 1, wherein etching a portion of the at least one silicon microbar to produce at least one silicon nanowire comprises
patterning a photoresist to define a central portion of the at least one microbar,
depositing a masking layer on the SOI starting wafer,
removing the photoresist to expose the central portion of the at least one microbar,
etching out the central portion of the at least one microbar, and
removing the mask.

7. The method of claim 6, wherein the mask comprises germanium.

8. The method of claim 6, wherein etching out the central portion of the at least one microbar comprises etching out the central portion of the at least one microbar using TMAH.

9. The method of claim 1, further comprising forming a back gate connection using photolithography.

10. The method of claim 1, further comprising forming a microchannel transverse to the at least one silicon nanowire.

11. A method of detecting a target analyte, comprising the steps of:
providing a silicon nanowire made using the method of claim 1;
sensitizing the silicon nanowire with a probe, wherein the probe is specific for a target analyte;
obtaining a first electrical measurement from the silicon nanowire;
exposing the probe to an unknown solution thought to contain the target analyte;
obtaining a second electrical measurement from the silicon nanowire; and
determining a change between the second measurement and the first measurement to detect the analyte.

12. The method of claim 11, wherein sensitizing the silicon nanowire with a probe comprises applying an electrically conductive coating to the silicon nanowire and coupling the probe to the electrically conductive coating.

13. The method of claim 11, wherein the electrically conductive coating comprises polyaniline.

14. The method of claim 11, wherein at least one of the first electrical measurement and the second electrical measurement comprises impedance.

15. The method of claim 11, wherein the probe comprises an antibody.

16. The method of claim 11, wherein the target analyte comprises bacteria.

17. The method of claim 11, wherein exposing the probe to an unknown solution thought to contain the target analyte comprises exposing the probe to an unknown solution thought to contain the target analyte using a microchannel in contact with the silicon nanowire.

18. A system for detecting a target analyte, comprising:
at least one silicon nanowire made using the method of claim 1, the at least one silicon nanowire having an electrically conductive coating thereon, the electrically conductive coating having a probe that is specific for a target analyte coupled thereto;
an electrical measurement system in communication with the at least one silicon nanowire; and
a microchannel transverse to the at least one silicon nanowire for introduction of a sample to the at least one silicon nanowire.

19. The system of claim 18, wherein the at least one silicon nanowire comprises a plurality of silicon nanowires, wherein each of the plurality of nanowires has a probe that is specific for a different target analyte coupled thereto.

20. The system of claim 11, wherein the microchannel is coupled to a microfluidic system.
